SEQUENCE LISTING (11 sequences)

SEQ ID NO: 1 - protein, 688 aa, Homo sapiens
  misc (1)..(688): Sequence for PPAR-gamma-2-gamma-2, a modified human PPAR-gamma (Peroxisome Proliferator Activated Receptor- gamma)
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLYAWAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 2 - DNA, 19 bp, Artificial Sequence
  PPAR binding site
tcaacctttaccctggtag

SEQ ID NO: 3 - DNA, 13 bp, Artificial Sequence
  PPAR binding site
aggtcaaaggtca

SEQ ID NO: 4 - DNA, 30 bp, Artificial Sequence
  primer
atgcatcgatggccgcttcgagcagacatg

SEQ ID NO: 5 - DNA, 39 bp, Artificial Sequence
  primer
atgcgtcgactctagccgattttaccacatttgtagagg

SEQ ID NO: 6 - DNA, 33 bp, Artificial Sequence
  primer
cgagcatgctgctgctgctgctgctgctgggcc

SEQ ID NO: 7 - DNA, 33 bp, Artificial Sequence
  primer
gggtctagattaacccgggtgcgcggcgtcggt

SEQ ID NO: 8 - DNA, 20 bp, Artificial Sequence
  primer
cgatcatgttcgacgacgcc

SEQ ID NO: 9 - DNA, 20 bp, Artificial Sequence
  primer
ccaggtcgcaggcggtgtag

SEQ ID NO: 10 - RNA, 23 nt, Artificial Sequence
  aptamer
ggccugggcgagaaguuuaggcc

SEQ ID NO: 11 - RNA, 72 nt, Artificial Sequence
  aptamer
ggugaucagauucugauccaauguuaugcuucucugccugggaacagcugccugaagcuuuggauccgucgc

(19) United States
(12) Patent Application Publication
(10) Pub. No.: US 2002/0166132 A1
(43) Pub. Date: Nov. 7, 2002

(54) System for regulating in vivo the expression of a transgene by conditional inhibition

(76) Inventors: Daniel Scherman, Paris (FR); Michael Bettan, Paris (FR); Pascal Bigey, Paris (FR)

Correspondence Address: Finnegan, Henderson, Farabow,, Garrett & Dunner, L.L.P., 1300 I Street, N. W., Washington, DC, 20005-3315, US

(21) Appl. No.: 09/931,007

(22) Filed: Aug. 17, 2001

Related U.S. Application Data
(60) Provisional application No. 60/239,246, filed on Oct. 11, 2000.

(30) Foreign Application Priority Data
Aug. 18, 2000 (FR) 001/10730

(51) Int. Cl. A01K 67/00; A61K 48/00

(52) U.S. Cl. 800/8; 800/13; 514/44

(57) ABSTRACT

The present invention relates to novel constructs and compositions and to a novel method for regulating the expression of a transgene of interest in vivo by conditional inhibition, and to the uses thereof in experimental, clinical and therapeutic domains or for the production of animals or plants.

For example, the novel regulation method is based on the coexpression of a transgene of interest encoding a transcript of interest and of an inhibitory transgene encoding an inhibitory transcript specific for the transcript of interest, so as to obtain constitutive inhibition of the activity of the transcript of interest, and to be able to ensure effective regulation of the transcript of interest, either by inhibiting its inhibitory transcript, or by activating the transcript of interest, or alternatively by activating the transcript of interest and concomitantly inhibiting its inhibitory transcript.

The present invention relates to novel compositions and to a novel method intended for controlling the expression in vivo of a transgene of therapeutic or experimental interest, using a system of conditional inhibition. The present invention is, for example, useful for generating modified animals and plants, and in gene therapy applications.

Gene therapy, which comprises correcting a deficiency or an abnormality (mutation, aberrant expression, etc.), or alternatively in treating a pathology, using the expression of a therapeutic transgene, is generally carried out by introducing an exogenous gene or transgene into the cell or tissue effected. The transgene is placed under the control of a strong promoter, constitutive or inducible, in order to ensure quantitatively and qualitatively optimal expression in vivo.

However, while these constitutive expression systems make it possible to obtain effective levels of expression of a transgene of interest which has been transferred, they do not offer the possibility of modulating the level of expression of the transgene. Moreover, in the case of current inducible systems, residual expression of the transgene of interest which is often too high, and which may cause a certain toxicity which is incompatible with a therapeutic or experimental use, is generally observed.

Now, the possibility of exerting effective control, for example of inhibition, of the transgene of interest may turn out to be determinant for the success of certain experiments or of the therapy, such as when the expression of the transgene is accompanied by side effects, for example cytotoxic side effects. This is generally the case for certain cytokines, such as TNF-α, IL-2, IL-4, IL-12, IL-18 or GM-CSF (Agha-Mohammadi et al., J. Clin. Invest., 105 (2000) 1173-1176), for anticlotting agents, for antibodies, for certain enzymatic activators of active substances (Springer et al., J. Clin. Invest., 105 (2000) 1161-1167), for molecules toxic for cancers, or for hormones.

Various artificial systems for controlling expression have been designed in the prior art. A first system uses a regulatory protein designated LAP (Lac Activator Protein) constructed by fusion of the E. coli Lac repressor with the transactivating domain of VP16 of the herpesvirus (HSV). LAP is capable of activating, in the absence of isopropyl β-D-thiogalactoside (IPTG), a minimum early promoter of SV40 which comprises, upstream or downstream of the transcription unit, the lac operator sequences, whereas in the presence of IPTG, the activation of the promoter is inhibited (Labow et al., Mol. Cell. Biol., 10 (1990) 3343-3356).

Another system uses a tetracycline-controlled transactivating protein, which has been constructed by fusion of the E. coli Tet repressor with the transactivating domain of VP16 of HSV, so as to activate, in the absence of tetracycline, the transcription from a minimum promoter comprising the tetracycline response tet operator sequences, this activation being able to be inhibited in the presence of tetracycline or of a derivative thereof (Gossen et al., Proc Natl Acad Sci USA, 89, (1992) 5547-5551; Gossen et al., Science, 268 (1995) 1766-1769).

These negative regulation systems suffer, however, from a residual expression which is still too high in the inhibited state, which limits their effectiveness and their uses in vivo. In addition, these systems require the provision of a repressor agent, such as tetracycline or IPTG, which is restrictive when only periodic expression of the transgene of interest is required.

Other systems for inhibiting the expression of genes, which use recombinant nucleic acids, such as antisense oligonucleotides (WO83/01451) or antisense RNAs which are complementary to an endogenous target gene (McCall, Biochim Biophys Acta, 1397(1) (1998), 65-72), have been developed.

They have to date only been used for regulating endogenous genes. Although they make it possible to obtain approximately 60 to 90% inhibition when they are tested in vitro, they are altogether ineffective for regulating endogenous genes in vivo, to such an extent that their development has been put aside, despite their low toxicity and the absence of immunogenicity.

Surprisingly, the applicants have discovered that, while the inhibition of an exogenous gene or transgene by a complementary antisense RNA in vitro is of the same order as that obtained with an antisense RNA complementary to an endogenous gene, i.e. very unsatisfactory, the inhibition of this same exogenous gene by its complementary antisense transcript is strong when it is carried out in vivo.

The applicants have, moreover, discovered that this inhibition is not reproduced by firstly injecting and expressing the transgene alone and then, secondly, injecting the sequence encoding its inhibitory transcript, but that, on the contrary, it is necessary to coinject and coexpress the nucleic acids comprising the sequences of the inhibitory antisense transcript and of the transgene, in order to obtain effective inhibition of the latter in vivo.

The applicants have finally discovered that the transgene can not only be effectively inhibited by its antisense RNA, but also that it is possible to re-establish a biologically effective level of expression of the transgene and thus to control the expression of the latter via its antisense-type specific inhibitory transcript.

### BRIEF DESCRIPTION OF THE DRAWINGS

FIGS. 1A to 1E: Schematic representations of plasmids pXL3031 (FIG. 1A), pXL3010 (FIG. 1B), pSeAPantisense (FIG. 1C), pXL3296 (FIG. 1D) and pLucAtisense (FIG. 1E).

FIGS. 2A to 2E: Schematic representations of plasmids pTet-Splice (FIG. 2A), pTetLucAntisense (FIG. 2B), pTetLuc (FIG. 2C), pTetSeAP antisense (FIG. 2D) and pTet-tTAk (FIG. 2E).

FIGS. 3A to 3D: Schematic representations of plasmids pGJA1 (FIG. 3A), pGJA2 (FIG. 3B), pGJA3 (FIG. 3C) and pGJA9 (FIG. 3D).

FIGS. 4A to 4D: Schematic representations of plasmids pGJA15-2 (FIG. 4A), pGJA15 (FIG. 4B), pGJA14 (FIG. 4C) and pGJA14-2 (FIG. 4D).

FIGS. 5A and 5B: Schematic representations of plasmids pRDA02 (5B) and pSG5-hPPARγ2 (5A).

FIGS. 6A to 6C: Schematic representation of plasmids pIND (6A), and pINDSeAP (6B), and pVgRXR (6C).

FIG. 7(A): Illustrates the activity of the SeAP measured 48 h after cotransfection of NIH3T3 cells with the following plasmids:

1: 0.25 μg of pXL3010 (S)+0.75 μg pXL3296 (V);

2: 0.25 μg of pXL3010 (S)+0.25 μg pSeAPantisense (A)+0.50 μg pXL3296 (V);

3: 0.25 μg of pXL3010 (S)+0.50 μg pSeAPantisense (A)+0.25 μg pXL3296 (V);

4: 0.25 μg of pXL3010 (S)+0.75 μg pSeAPantisense (A); and

5: 0.25 μg of pSeAPantisense (A)+0.75 μg pXL3296 (V).

FIG. 7(B): Illustrates the luciferase relative activities measured 24 h after cotransfection of the following plasmids:

1: 0.125 μg of pXL3031+0.75 μg pXL3296.

2: 0.125 μg of pXL3031+0.125 μg pLucAntisense+0.25 μg pXL3296.

3: 0.125 μg of pXL3031+0.25 μg pLucAntisense+0.125 μg pXL3296.

4: 0.125 μg of pXL3031+0.375 μg pLucAntisense.

5: 0.125 μg of pLucAntisense+0.375 μg pXL3296.

FIG. 8: Represents a photograph of an electrophoresis gel illustrating the presence of the sense and antisense RNAs by RT-PCR in vitro.

Lanes 1 and 9: 100-base pair marker (Gibco BRL)

Lane 2: PCR control using the plasmid pXL3010 as a matrix.

Lane 3: RT-PCR on the total RNAs extracted from the cells transfected with 0.25 μg of pXL3010+0.75 μg pXL3296.

Lane 4: RT-PCR on the RNAs extracted from the cells transfected with 0.25 μg of pXL3010+0.25 μg pSeAPantisense+0.50 μg pXL3296.

Lane 5: RT-PCR on the RNAs extracted from the cells transfected with 0.25 μg of pXL3010+0.75 μg pSeAPantisense.

Lanes 6 to 8: PCR controls (without RT) performed on the RNAs used in 3, 4 and 5, respectively.

FIG. 9A: Illustrates the SeAP activities in vitro measured 24 h after cotransfection of the following sets of plasmids:

Condition 1: 25% pXL3010+75% pXL3296

Condition 3: 25% pXL3010+25% pSeAPantisense+50% pXL3296

Condition 5: 25% pXL3010+25% pLucAntisense+50% pXL3296

FIG. 9B: Illustrates the luciferase relative activities measured 24 h subsequent to independent transfections in vitro of the following sets of plasmids:

Condition 2: 25% pXL3031+75% pXL3296

Condition 4: 25% pXL3031+25% pLucantisense+50% pXL3296

Condition 6: 25% pXL3031+25% pSeAPantisense+50% pXL3296

FIG. 10: Illustrates the relative levels of circulating SeAP measured after bilateral intramuscular injections into the tibialis cranialis skeletal muscle and electrotransfer of plasmids encoding the sense sequence (pXL3010) and the antisense sequence (pSeAPantisense) of the SeAP reporter gene, either simultaneously (batch 2) or 22 days apart (batch 1).

Batch 1: 10 mice injected with 30 μg of a plasmid pXL3010+electrotransfer, then injection of 30 μg of pSeAPantisense+electrotransfer (2nd injection on day 22);

Batch 2: 10 mice coinjected with 30 μg of a plasmid pXL3010+30 μg of a plasmid pSeAPantisense+electrotransfer (coinjection);

Batch 3: 10 mice injected with 30 μg of a plasmid pSeAPantisense+electrotransfer (control group).

FIG. 11A: Represents a photograph of an electrophoresis gel illustrating the presence of sense and antisense RNAs of the SeAP reporter gene by RT-PCR in vivo of batches 1 to 3 of FIG. 6

Lane 1 and 13: 100-bp DNA marker (Gibco);

Lane 2 and 3: sense and antisense RNA, respectively, in muscles of the mice of batch 1 (pXL3010, then reinjection of pSeAPantisense 22 days later);

Lanes 4 and 5: sense and antisense RNA, respectively, in muscles of the mice of batch 2 (coinjection of pXL3010 and of pSeAPantisense);

Lanes 6 and 7: sense and antisense RNA, respectively, in muscles of the mice of batch 3 (pSeAPantisense alone).

Lanes 8 to 10: PCR controls without RT, of the RNAs used in lanes 2 to 7;

Lane 11: control: PCR using the plasmid pXL3010 as a matrix;

Lane 12: plasmid pXL3010.

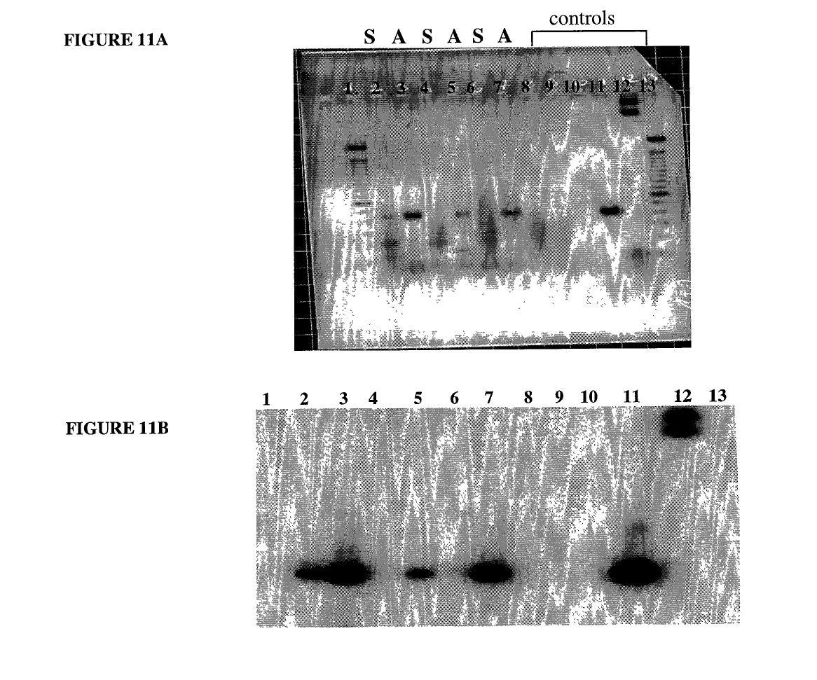

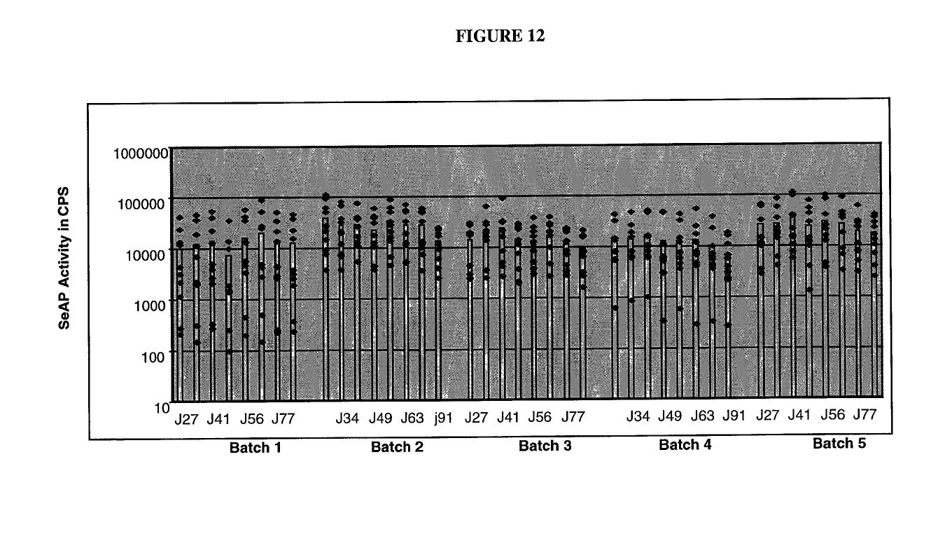

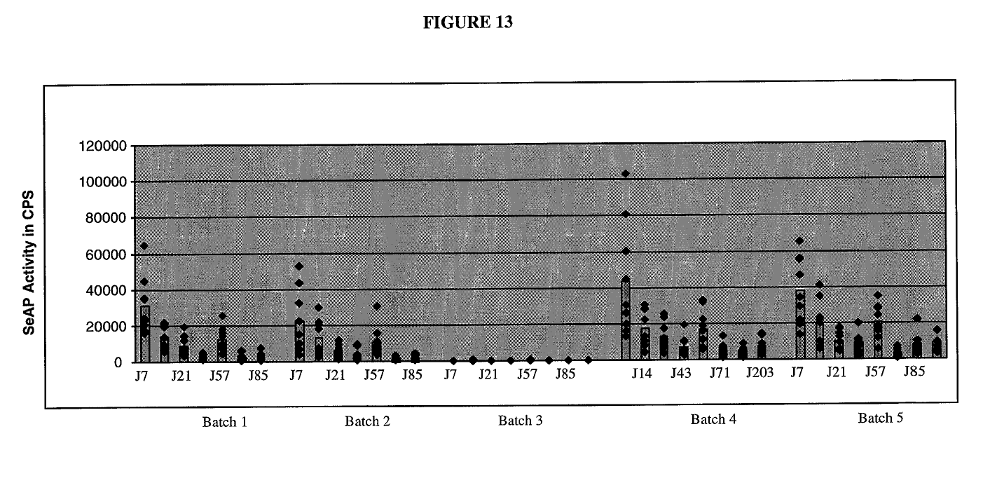

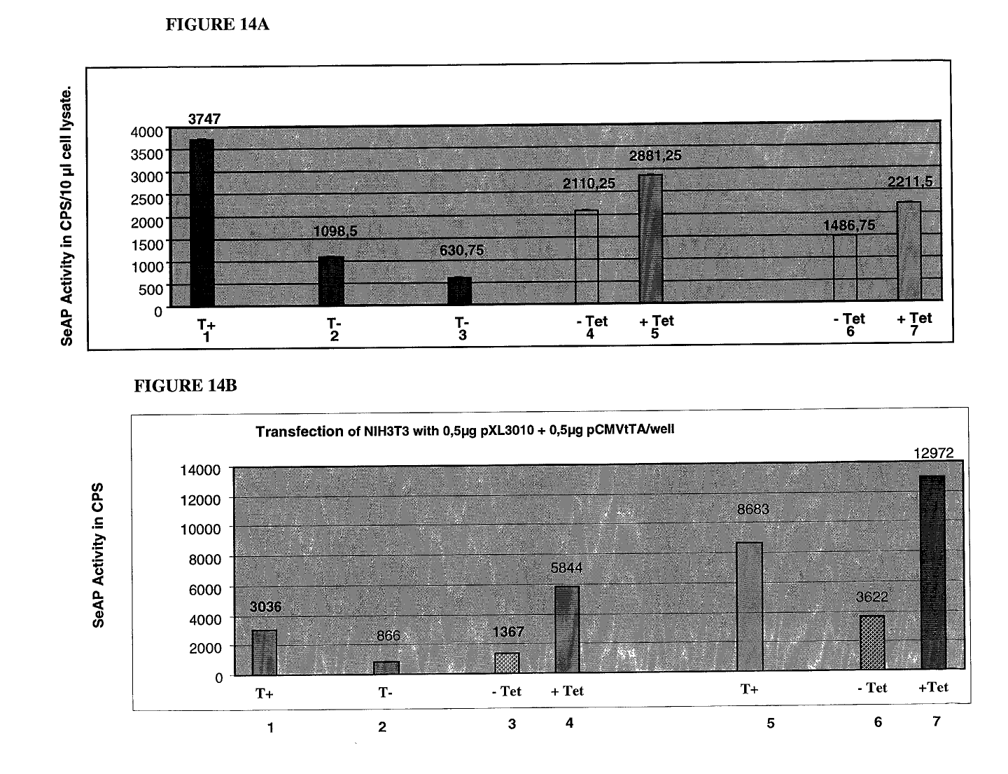

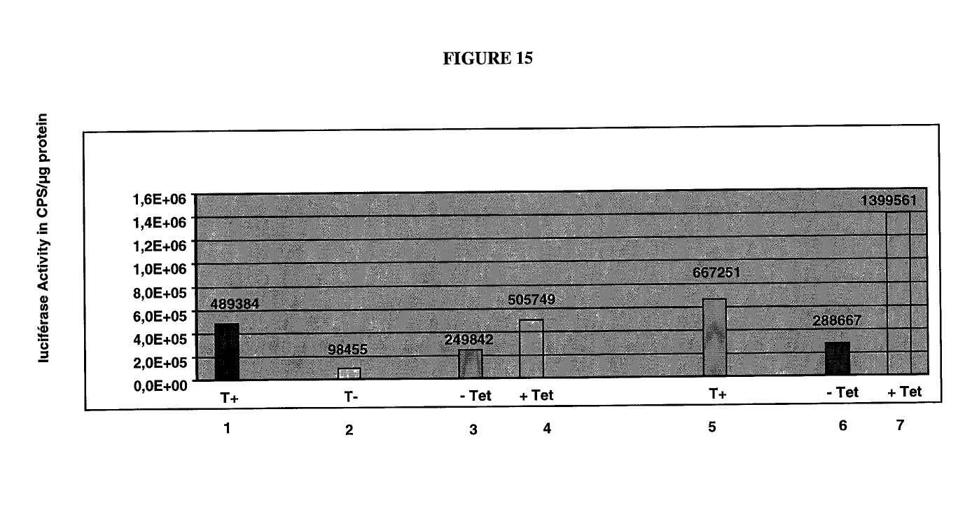

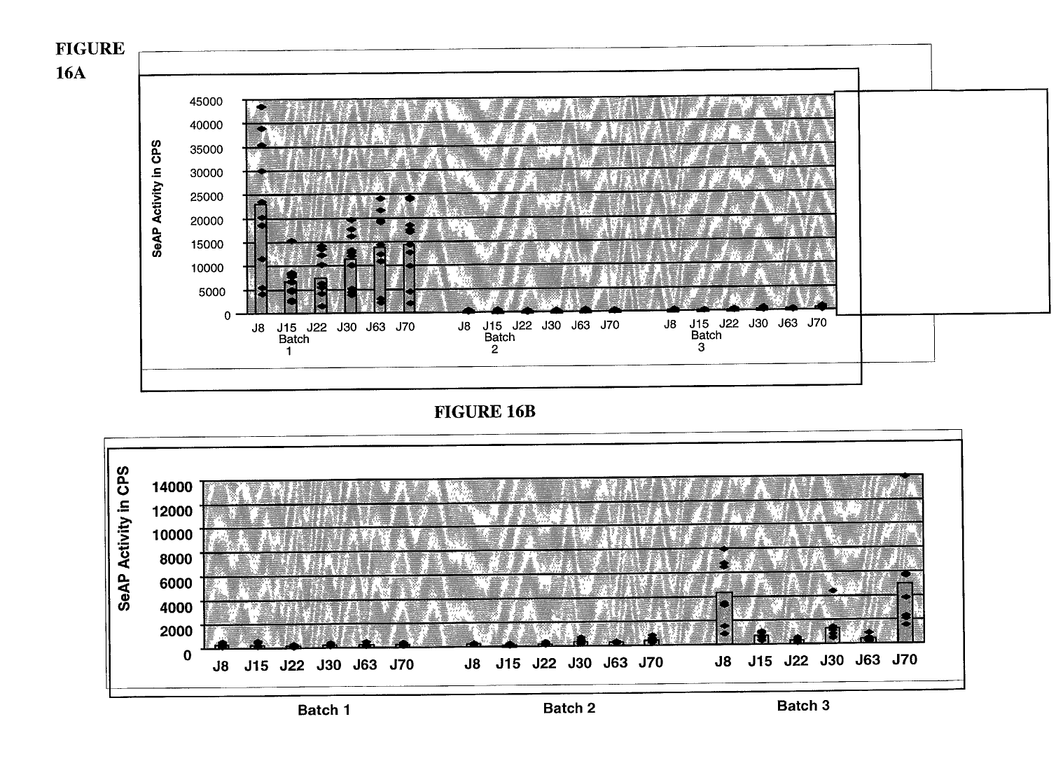

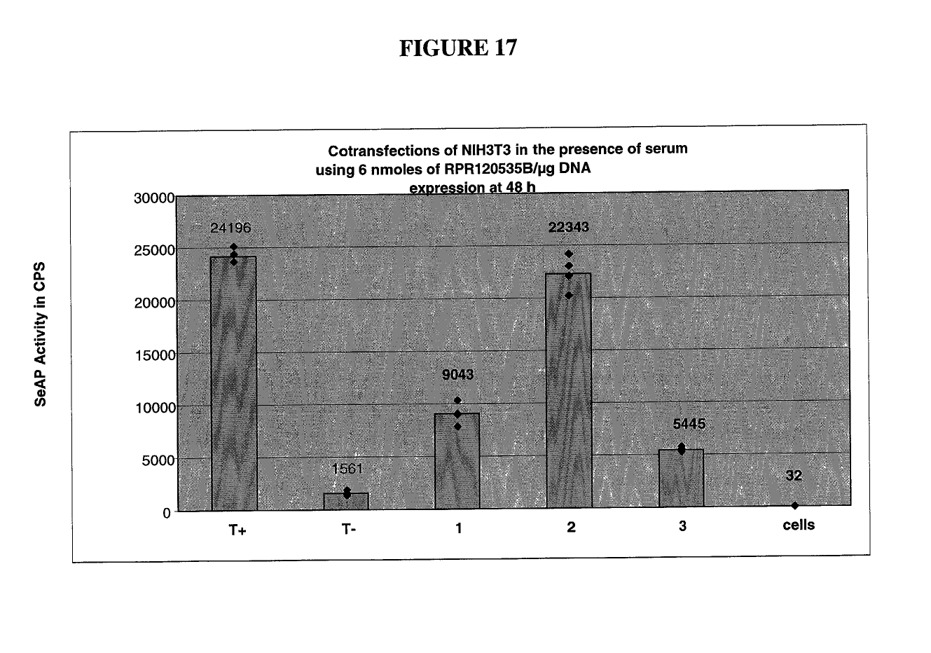

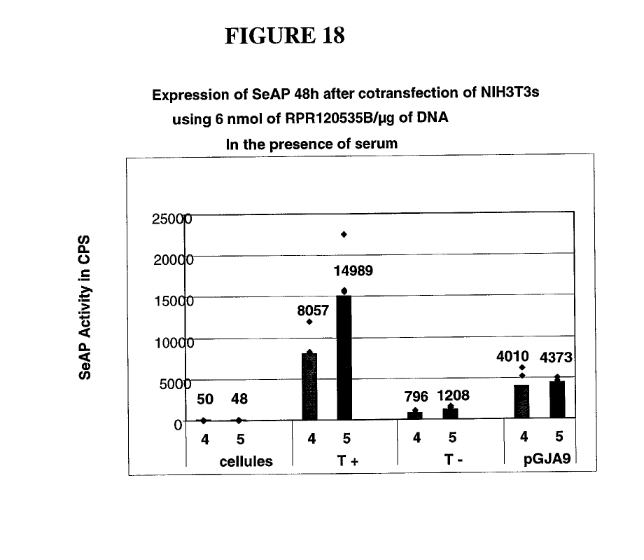

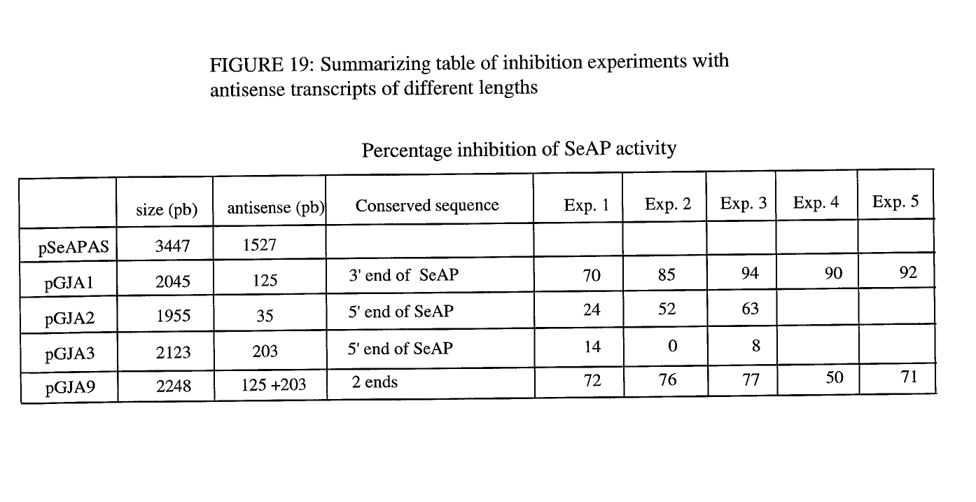

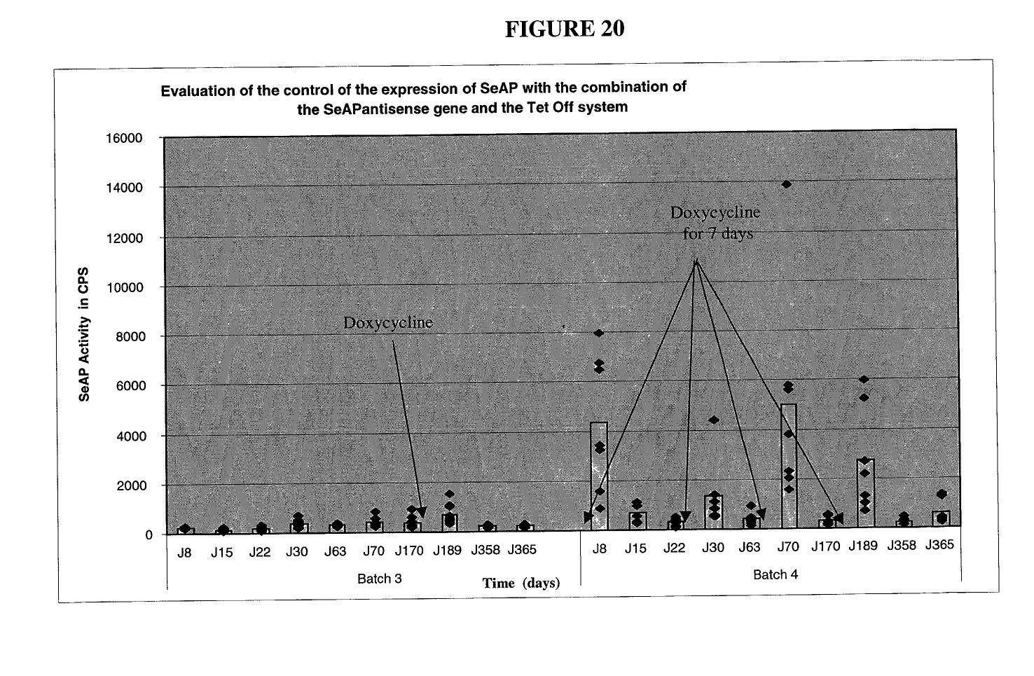

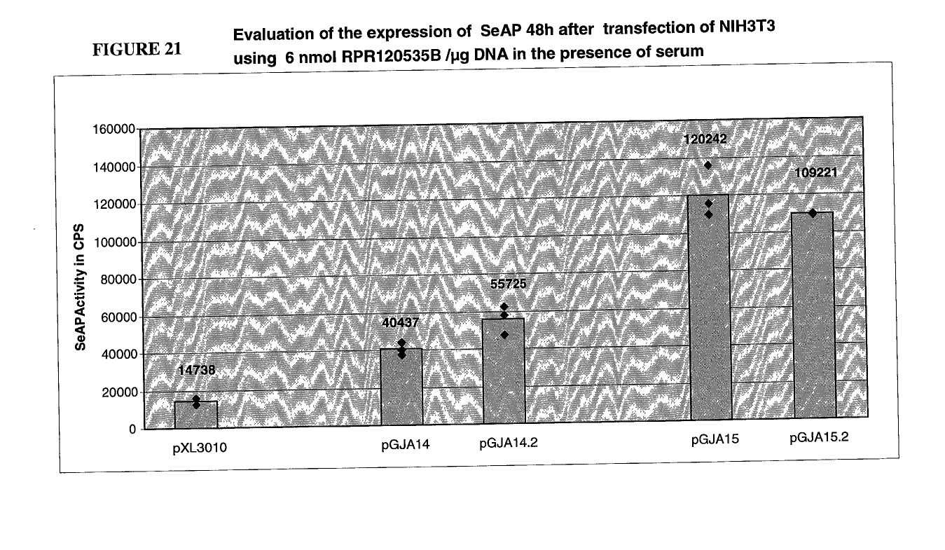

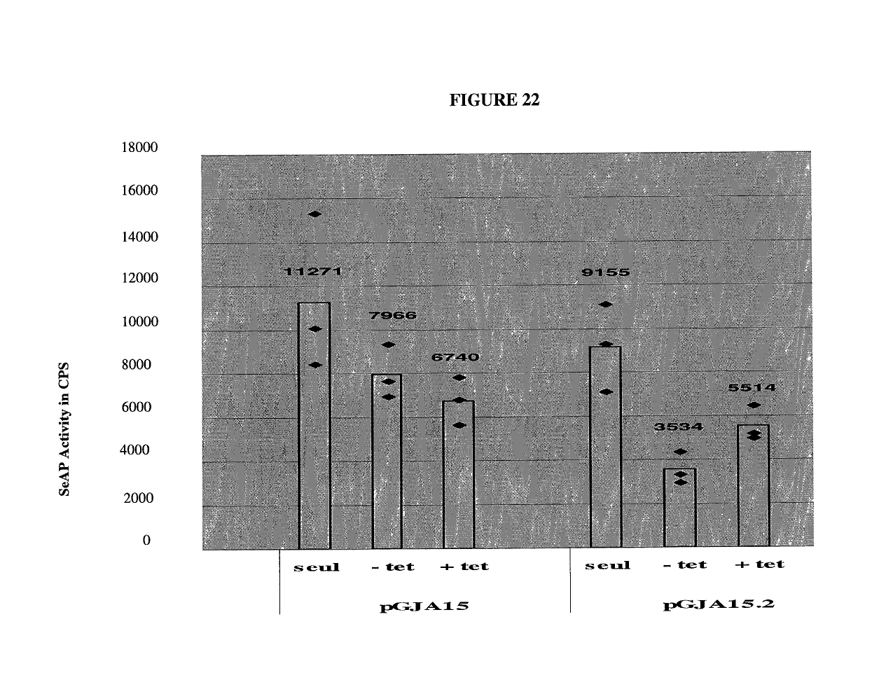

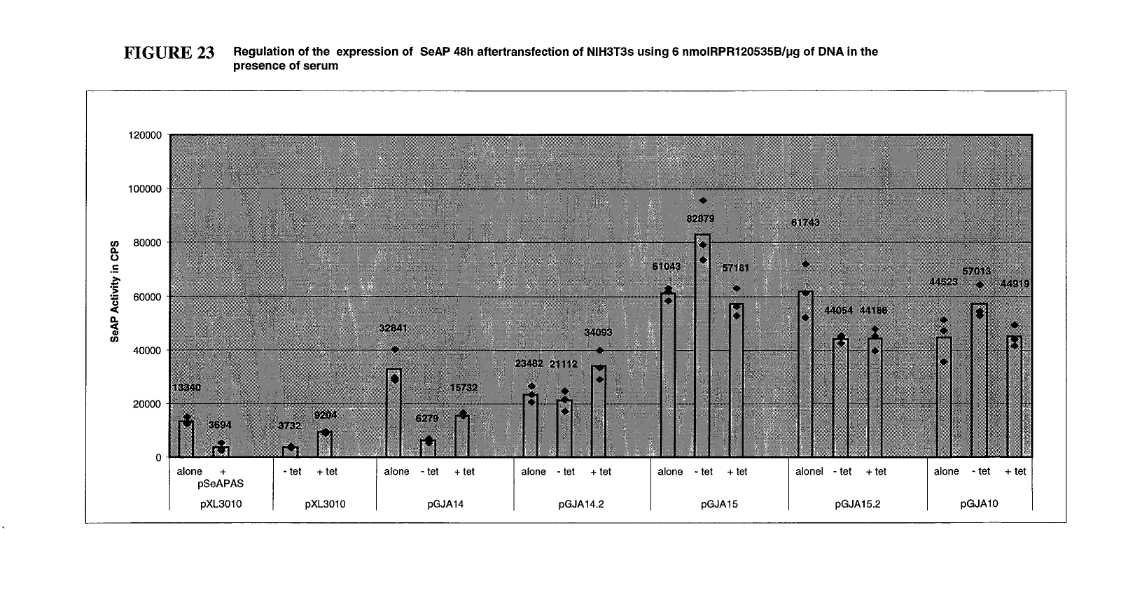

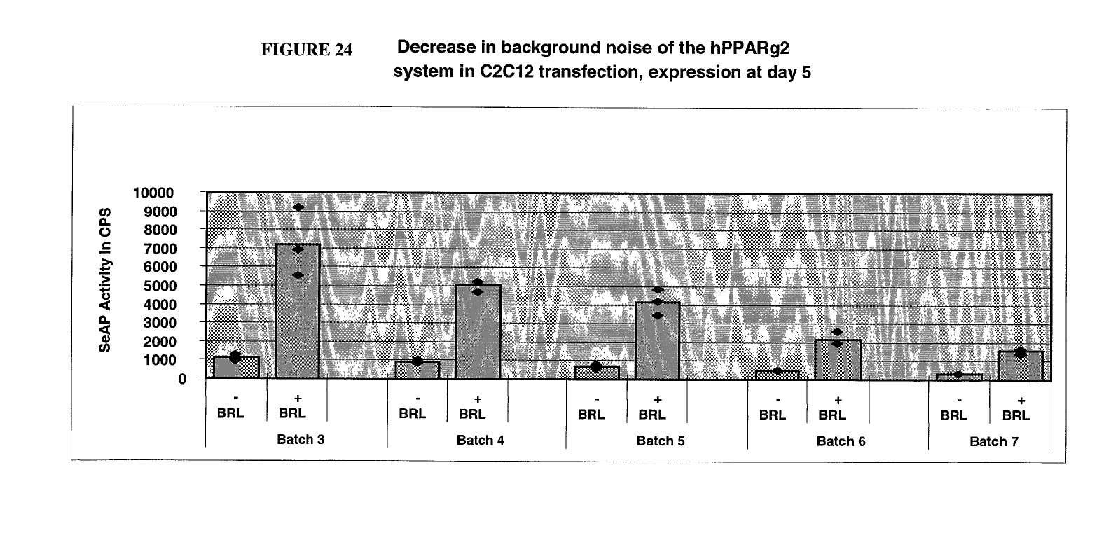

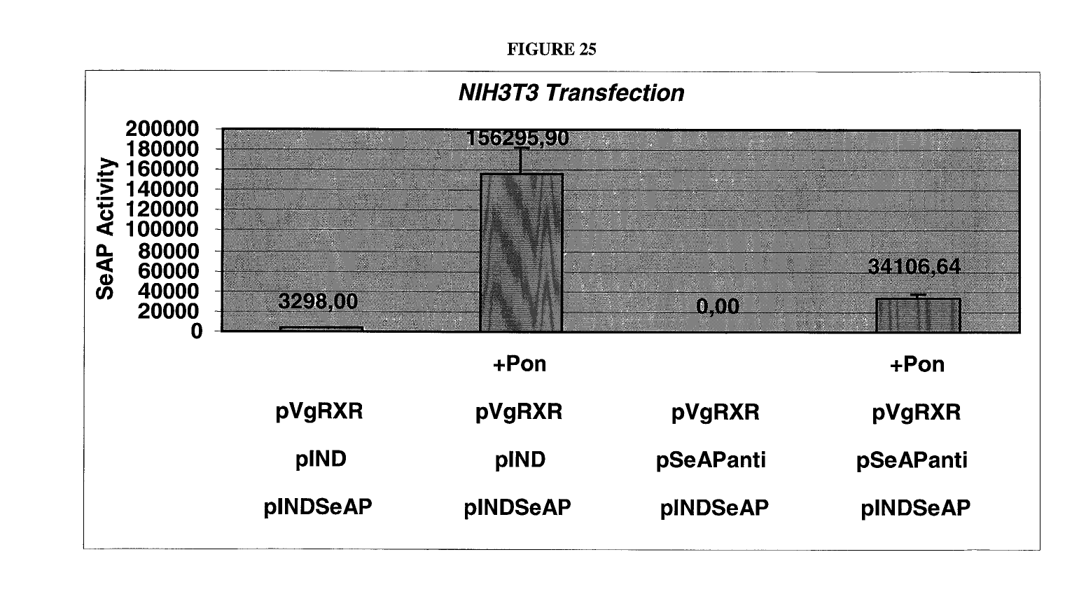

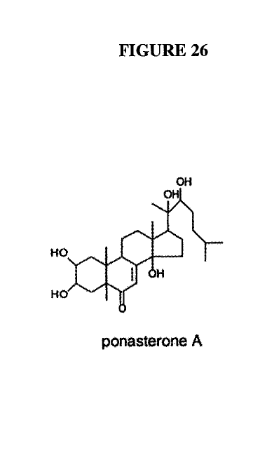

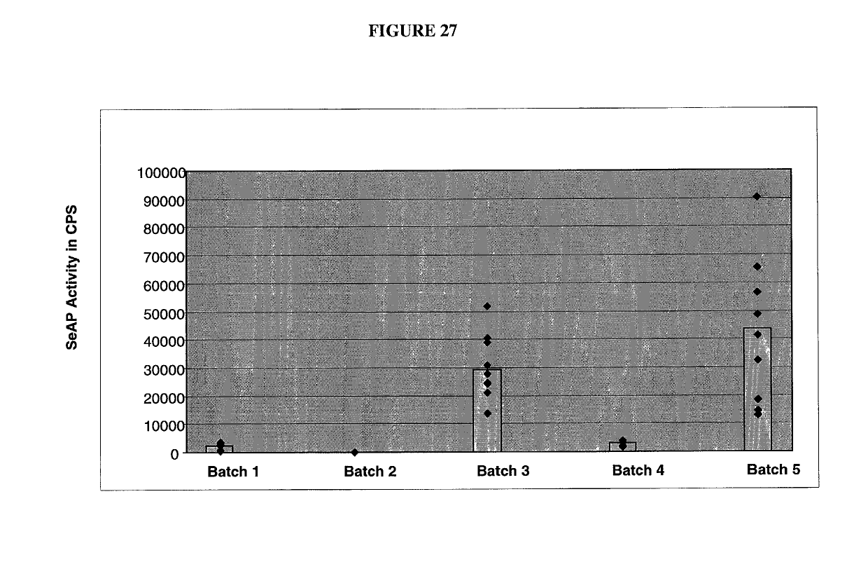

FIG. 11B: Represents a photograph of an X-ray film obtained by transfer and hybridization on a nitrocellulose membrane of the agarose gel photographed in FIG. 11A, in the presence of ³²P-labelled oligonucleotide probes specific for the sense sequence of the SeAP reporter gene (S) and of the antisense sequence (AS).

FIG. 12: Monitoring of the relative activity of circulating SeAP in the mouse plasma after bilateral intramuscular injections into the tibialis cranialis skeletal muscle and electrotransfer of the following plasmids at the time intervals described below:

Batch 1: 10 mice injected with 15 μg of plasmid pXL3010+electrotransfer.

Batch 2: 10 mice injected with 15 μg of plasmid pXL3010+electrotransfer, then injection of 45 μg of pXL3296+electrotransfer 21 days later;

Batch 3: 10 mice injected with 15 μg of plasmid pXL3010+electrotransfer, then injection of 15 μg of pSeAPantisense+30 μg of pXL3296+electrotransfer 21 days later;

Batch 4: 10 mice injected with 15 μg of plasmid pXL3010+electrotransfer, then injection of 30 μg of pSeAPantisense+15 μg of pXL3296+electrotransfer 21 days later;

Batch 5: 10 mice injected with 15 μg of plasmid pXL3010+electrotransfer, then injection of 45 μg of pSeAPantisense+electrotransfer 21 days later.

FIG. 13: Monitoring of the relative activity of circulating SeAP in the mouse plasma after coinjection and electrotransfer (ET) of the following plasmids:

Batch 1: 9 mice injected with 30 μg of plasmid pXL3010+ET;

Batch 2: 9 mice injected with 30 μg of plasmid pXL3010+ET;

Batch 3: 9 mice coinjected with 30 μg of plasmid pXL3010+30 μg of pSeAPantisense+ET:

Batch 4: 9 mice injected with 30 μg of plasmid pXL3010+ET;

Batch 5: 9 mice injected with 30 μg of plasmid pXL3010+ET.

FIG. 14A: Relative activities of SeAP in vitro measured after transfection of NIH3T3 cells with the following plasmids, with or without subsequent tetracycline treatment:

Column 1: 1 μg pXL3010+1 μg pXL3296 (empty)

Column 2: 1 μg pXL3010+0.5 μg pXL3296 (empty)+0.5 μg pSeAPantisense

Column 3: 1 μg pXL3010+1 μg pSeAPantisense

Column 4: 1 μg pXL3010+0.5 μg pTetSeAPantisense+0.5 μg pTet-tTAk without tetracycline

Column 5: 1 μg pXL3010+0.5 μg pTetSeAPantisense+0.5 μg pTet-tTAk with tetracycline (1 mg/ml)

Column 6: 1 μg pXL3010+1 μg pTetSeAPantisense+0.5 μg pTet-tTAk without tetracycline

Column 7: 1 μg pXL3010+1 μg pTetSeAPantisense+0.5 μg pTet-tTAk with tetracycline (1 mg/ml)

FIG. 14B: Relative activities of SeAP in vitro measured after transfection of NIH3T3 cells with the following plasmids, with or without subsequent tetracycline treatment:

Column 1: 0.5 μg pXL3010+0.5 μg pTet-tTAk+0.5 μg pXL3296 (empty)

Column 2: 0.5 μg pXL3010+0.5 μg pTet-tTAk+0.5 μg pSeAPantisense

Column 3: 0.5 μg pXL3010+0.5 μg pTet-tTAk+0.5 μg pTetSeAPantisense without tetracycline

Column 4: 0.5 μg pXL3010+0.5 μg pTet-tTAk+0.5 μg pTetSeAPantisense with tetracycline (1 mg/ml)

Column 5: 0.5 μg pXL3010+2.5 μg pXL3296 (empty)

Column 6: 0.5 μg pXL3010+0.5 μg pTet-tTak+2.5 μg pTetSeAPantisense without tetracycline

Column 7: 0.5 μg pXL3010+0.5 μg pTet-tTak+2.5 μg pTetSeAPantisense with tetracycline (1 mg/ml)

FIG. 15: Luciferase relative activities 24 h after cotransfection of the NIH 3T3 cells (80 000 cells per well) with the following plasmids (0.7 or 1.1 μg of DNA per well), with or without administration of tetracycline:

1: 0.1 μg pXL3031+0.3 μg pTet-tTAk+0.3 μg pXL3296.

2: 0.1 μg pXL3031+0.3 μg pTet-tTAk+0.3 μg pLucAntisense.

3: 0.1 μg pXL3031+0.3 μg pTet-tTAk+0.3 μg pTetLucAntisense without tetracycline.

4: 0.1 μg pXL3031+0.3 μg pTet-tTAk+0.3 μg pTetLucAntisense with tetracycline (1 mg/ml).

5: 1 μg pXL3031+0.5 μg pTet-tTAk+0.5 μg pXL3296.

6: 0.1 μg pXL3031+0.5 μg pTet-tTAk+0.5 μg pTetLucAntisense without tetracycline.

7: 0.1 μg pXL3031+0.5 μg pTet-tTAk+0.5 μg pTetLucAntisense with tetracycline (1 mg/ml).

FIG. 16A: Relative levels of circulating SeAP in vivo after intramuscular coinjection into 6-week-old female SCID mice of the following plasmids, with or without administration of tetracycline at varying time intervals:

Batch 1: 10 mice injected with 20 μg of plasmid pXL3010+40 μg pTet-tTAk;

Batch 2: 10 mice injected with 20 μg of plasmid pXL3010+20 μg pTet-tTAk+20 μg pSeAPantisense;

Batch 3: 10 mice injected with 20 μg of plasmid pXL3010+20 μg pTet-tTAk+20 μg pTetSeAPantisense.

FIG. 16B: Relative levels of circulating SeAP in vivo after intramuscular coinjection into 6-week-old female SCID mice of the following plasmids, with or without administration of tetracycline at varying time intervals:

Batch 1: 10 mice injected with 20 μg of plasmid pXL3010+20 μg pTet-tTAk+20 μg pSeAPantisense;

Batch 2: 10 mice injected with 20 μg of plasmid pXL3010+20 μg pTet-tTAk+20 μg pTetSeAPantisense;

Batch 3: Batch 2+tetracycline-comprising drink (2 mg/ml+2 mg/ml of sucrose) for 9 days, then tetracycline stopped on the 10th day. Put back on tetracycline on the 22nd day (IP injection every two days, 500 μg/mouse), and stopped on the 30th day. Put on doxycycline on the 63rd day (400 mg/l in the drink).

FIG. 17: Measurement of the expression of SeAP measured 48 h after cotransfection of NIH3T3 cells with the following plasmids:

T+: 1 μg pXL3010+1 μg pXL3296

T−: 1 μg pXL3010+1 μg pSeAPantisense

1: 1 μg pXL3010+1 μg pGJA1

2: 1 μg pXL3010+1 μg PGJA2

3: 1 μg pXL3010+1μg pGJA3

FIG. 18: Measurement of the expression of SeAP measured 48 h after cotransfection of NIH3T3 cells with the following plasmids: Columns 1 and 2: control of nontransfected cells, two distinct experiments termed 4 and 5;

T+: 1 μg pXL3010+1 μg pXL3296, experiments 4 and 5, resepctively;

T−: 1 μg pXL3010+1 μg pSeAPantisense, experiments 4 and 5, respectively; PGJA9: 1 μg pXL3010+1 μg pXL3296+1 μg pXGJA9, experiments 4 and 5, respectively.

FIG. 19: Summarizing table of the inhibitions of SeAP expression obtained by transfecting the plasmids pGJA1, pGJA2, pGJA3 and pGJA9 into NIH3T3 cells, compared with the inhibition produced by the plasmid comprising the entire antisense sequence SeAPantisense.

FIG. 20: Monitoring of the relative activity of circulating SeAP in the plasma of mice after bilateral intramuscular injections into the tibialis cranialis skeletal muscle and electrotransfer of the following plasmids, followed by administration of doxycycline at the following time intervals:

Batch 3: a batch of mice injected with 20 μg pXL3010+20 μg pTet-tTAk+20 μg pTetSeAPantisense, and 400 mg/l doxycycline added only on day 170;

Batch 4: a batch of mice injected with 20 μg pXL3010+20 μg pTet-tTAk+20 μg pTetSeAPantisense, and 400 mg/l doxycycline for 7-day periods at the periods of time indicated.

FIG. 21: Measurement of the expression of SeAP measured 48 h after transfection of NIH3T3 cells with the following plasmids, for a copy number equivalent to 1 μg pXL3010, qs for pXL3296:

Column 1: pGJA14;

Column 2: pGJA14-2;

Column 3: pGJA15; and

Column 4: pGJA15-2.

FIG. 22: Measurement of the expression of SeAP measured 24 h after cotransfection of NIH3T3 cells with the following plasmids, for a copy number equivalent to 0.5 μg pXL3010, qs for pXL3296:

Column 1: pGJA15;

Column 2: pGJA15+pTet-tTAk

Column 3: pGJA15+pTet-tTAk+tetracycline 1 μg/ml final;

Column 4: pGJA15-2;

Column 5: pGJA15-2+pTet-tTAk;

Column 6: pGJA15-2+pTet-tTAk+tetracycline 1 μg/ml final.

FIG. 23: Measurement of the expression of SeAP measured 48 h after transfection of NIH3T3 cells with the following plasmids, for a copy number equivalent to 0.5 μg pXL3010, qs for pXL3296:

Column 1: pXL3010;

Column 2: pXL3010+pSeAPantisense;

Column 3: pXL3010+pTet-tTAk;

Column 4: pXL3010+pTet-tTAk+tetracycline 1 μg/ml final;

Column 5: pGJA14;

Column 6: pGJA14+pTet-tTAk;

Column 7: pGJA14+pTet-tTAk+tetracycline 1 μg/ml final.

Column 8: pGJA14.2;

Column 9: pGJA14.2+pTet-tTAk;

Column 10: pGJA14.2+pTet-tTAk+tetracycline 1 μg/ml final.

Column 11: pGJA15;

Column 12: pGJA15+pTet-tTAk;

Column 13: pGJA15+pTet-tTAk+tetracycline 1 μg/ml final.

Column 14: pGJA15.2;

Column 15: pGJA15.2+pTet-tTAk;

Column 16: pGJA15.2+pTet-tTAk+tetracycline 1 μg/ml final.

Column 17: pGJA10;

Column 18: pGJA10+pTet-tTAk;

Column 19: pGJA10+pTet-tTAk+tetracycline 1 μg/ml final.

FIG. 24: Measurement of the expression of SeAP 5 days after transfection in C2C12 cells with the following plasmids, with and without the chemical inducer BRL49653 at 10-7 M final:

Batch 3: 500 ng of pRDA02+500 ng pSG5-hPPARγ2+pXL3296 (column 1: without BRL49653; column 2: with BRL49653)

Batch 4: batch 3+50 ng pSeAPAS (column 3: without BRL49653; column 4: with BRL49653)

Batch 5: batch 3+100 ng pSeAPAS (column 5: without BRL49653; column 6: with BRL49653)

Batch 6: batch 3+250 ng pSeAPAS (column 7: without BRL49653; column 8: with BRL49653)

Batch 7: batch 3+500 ng pSeAPAS.

FIG. 25: Measurement of the expression of SeAP measured 48 h after transfection of NIH3T3 cells with the following plasmids, with and without the chemical inducer for the ecdysone system, Ponasterone or Pon (FIG. 26; No et al., PNAS, 1996, 93:3346-3351). Column 1: 0.5 μg of each plasmid pVgRXR, pIND, pINDSeAP, without chemical inducer;

Column 2: 0.5 μg of each plasmid pVgRXR, pIND, pINDSeAP, with chemical inducer;

Column 3: 0.5 μg of each plasmid pVgRXR, pINDSeAP, pSeAPantisense, without chemical inducer;

Column 4: 0.5 μg of each plasmid pVgRXR, pINDSeAP, pSeAPantisense, with chemical inducer.

FIG. 26: Representation of Ponasterone (pon)

FIG. 27: Monitoring of the relative activity of circulating SeAP, assayed using the Phospha Light kit (Tropix), in the plasma of mice after bilateral intramuscular injections into the tibialis cranialis skeletal muscle and electrotransfer of the following plasmids, with or without administration of doxycycline in the drinking water:

Batch 1: a batch of mice injected with 20 μg pXL3010+20 μg pcDNA;

Batch 2: a batch of mice injected with 20 μg pGJA14+20 μg pTet-tTAk;

Batch 3: a batch of mice injected with 20 μg pGJA14+20 μg pTet-tTAk+400 mg/ml of doxyclycline in the drink;

Batch 4: a batch of mice injected with 20 μg pGJA15-2+20 μg pTet-tTAk;

Batch 5: a batch of mice injected with 20 μg pGJA15-2+20 μg pTet-tTAK+400 mg/ml of doxycycline in the drink.

A subject of the present invention is a novel method for regulating in vivo the expression of a transgene of interest, comprising:

simultaneously introducing into a target tissue or cell a nucleic acid comprising the sequence of a transgene of interest encoding a transcript of interest or useful transcript, and a nucleic acid comprising the sequence of an inhibitory transgene encoding an inhibitory transcript specific for said transcript of interest, said sequences each being under the control of a transcriptional promoter, and the activity of the inhibitory transcript and/or of the transcript of interest possibly being regulated with an external agent, and

coexpressing said nucleic acids in the target tissue or cell in order to allow the constitutive inhibition of the activity of the transcript of interest with the inhibitory transcript.

Additionally, an external agent termed repressor may be optionally administered to the target tissue or cell, causing the activity of the inhibitory transcript to be inhibited, and thus activity of the transcript of interest to be restored, proportionally to the amount of the external repressor agent used.

Alternatively or additionally, an external agent termed an activator is administered to the target tissue or cell, causing the activity of the transcript of interest to be increased. Thus, activity of the transcript of interest can be restored proportionally to the amount of the external activator agent used.

A subject of the present invention is also a method for transferring in vivo a transgene of interest, comprising coadministering and coexpressing in a target tissue or cell a nucleic acid comprising the sequence of a transgene of interest encoding a transcript of interest or useful transcript, and a nucleic acid comprising the sequence of an inhibitory transgene encoding an inhibitory transcript specific for said transcript of interest. According to this method, the expression of the transgene of interest or the activity of the transcript of interest is inhibited constitutively and can be restored by inhibiting the activity of the inhibitory transcript, by administering an external repressor agent, and/or by administering an external agent capable of causing the induction of the activity of the transcript of interest.

A subject of the present invention is also a method intended for decreasing the residual expression of a transgene of interest in vivo, which comprises coinjecting and in coexpressing the sequences encoding the transcript of interest and its specific inhibitory transcript.

A subject of the present invention is also a novel combination administered in vivo and capable of being used in the method according to the invention. This combination includes a nucleic acid comprising the sequence of a transgene of interest encoding a transcript of interest or useful transcript, and a nucleic acid comprising a sequence of an inhibitory transgene encoding an inhibitory transcript specific for the transcript of interest, each of the sequences being under the control of a transcriptional promoter, and the activity of the transcript of interest and/or of the inhibitory transcript possibly being regulated with an external agent.

The term “transgene of interest” is intended to mean any exogenous nucleic acid molecule encoding a biological product, namely either a transcript of interest or useful transcript such as an mRNA, an rRNA, a tRNA, a ribozyme or an aptazyme, or a protein, a polypeptide or a peptide of therapeutic or experimental interest. According to the invention, the transgene of interest includes a gDNA, a cDNA or DNAs which are natural or obtained totally or partially by chemical synthesis.

The term “transcript of interest” or “useful transcript” is intended to mean an RNA produced by transcription from the transgene of interest as defined above. The transcript of interest can be in the form of an mRNA and be translated into a therapeutic protein or peptide with intracellular or secreted action. Alternatively, the transcript of interest or useful transcript can be in the form of an RNA which has intrinsic biological activity, such as an aptazyme, a ribozyme or an antisense RNA, or an RNA which is capable of interacting with the components of the transfected cells, such as for example a ribosomal RNA (rRNA), a transfer RNA (tRNA) or an aptamer.

The term “inhibitory transgene” is intended to mean any exogenous nucleic acid molecule capable of producing, by transcription, an inhibitory transcript which has the transcript of interest as its target. According to the invention, the inhibitory transgene includes a gDNA, a cDNA and DNAs which are natural or obtained totally or partially by chemical synthesis.

The term “specific inhibitory transcript” is intended to mean an RNA which can be in the form of an antisense RNA, of a ribozyme or of an RNA capable of forming a triple helix, and which has a certain complementarity with, or specificity for, the transcript of interest.

The transcript is termed inhibitory in so far as it is capable of effectively and constitutively inhibiting the transcript of interest, with which it is coexpressed in the target tissue or cell, either at the translational level, by blocking the translation of the transcript of interest of mRNA type, or at the level of its biological activity, by blocking the interaction of the rRNA, tRNA or aptamer transcript of interest with the cellular components, or by blocking the interaction of the transcript of interest of aptazyme, ribozyme or antisense RNA type with a target nucleic acid sequence, or alternatively by decreasing the concentration of the transcript of interest by enzymatic degradation. This inhibitory transcript is, moreover, termed repressible, i.e., it can itself be the object of inhibition via an external repressor agent.

The expression “activity of the transcript of interest” is intended to mean either its translation into a protein or peptide of therapeutic or experimental interest when the transcript of interest is in the form of an mRNA, or its biological activity when the transcript of interest is in the form of an aptazyme, of a ribozyme or of an antisense RNA, or alternatively its interaction with the cellular components, when the transcript of interest is in the form of a ribosomal RNA, of a transfer RNA or of an aptamer.

The term “external agent” is intended to mean any chemical agent, for example a pharmacological agent, or physical agent such as heat, which can be administered enterally or parenterally, which has a low toxicity, and which has activity for inhibiting or for activating the expression of a gene.

One of the advantageous characteristics of the method of regulation by reversible inhibition according to the present invention lies in its capacity to effectively block, in a constitutive manner, the expression of a transgene of interest in vivo or the activity of the transcript of interest or useful transcript, and to re-establish this expression when this is desired for clinical or experimental reasons. This system is based on the coinjection and coexpression of a transgene of interest and of its specific inhibitory transcript in vivo, and the possibility of effectively regulating the transgene of interest either by inhibiting its specific inhibitory transcript, or by activating the transcript of interest, or alternatively by activating the transcript of interest and concomitantly inhibiting its specific inhibitory transcript.

According to a first embodiment of the present invention, the inhibitory transcript is inhibited with an external repressor agent in order to lift the inhibition of the transcript of interest and to indirectly re-establish the activity of the transcript of interest or a sufficient biological level of the transcript of interest.

The inhibition of the inhibitory transcript can be obtained by placing the sequence of the inhibitory transgene encoding the inhibitory transcript under the control of a promoter which is repressible or sensitive to an external repressor agent. It is possible to use, for example, the tetracycline-mediated regression system (TrRS) which is derived from the E. coli tetracycline resistance operon (Gossen et al., Proc. Natl. Acad. Sci., 89 (1992), 5547-5551). This system uses the affinity of the tet repressor (tetR) for the sequence of the tet operator (tetO), the affinity of tetR for tetracycline, and the ubiquitous activity of the VP16 herpesvirus transactivator in eukaryotic cells. This TrRS regulation system therefore functions using a chimeric transactivator (tTA) which results from the fusion of the C-terminal end of VP16 with the C-terminal end of the tetR protein.

In the absence of tetracycline, the tetR portion of the tTA transactivator binds to a regulatory sequence which comprises, for example, repeat sequences (2, 7 or 10 repeats) of the tetracycline operator, and which is placed upstream of a minimum transcriptional promoter, for example, of the human cytomegalovirus (hCMV), and activates the transcription of the inhibitory transgene and the production of the inhibitory transcript, ensuring effective constitutive inhibition of the transcript of interest. In the presence of tetracycline, this binds to the tetR portion of the tTA chimeric transactivator and causes a change in its conformation and loss of affinity for the repeat sequences of the tetracycline response operator (tetO). Inhibition of the production of the inhibitory transcript from the inhibitory transgene, and the reestablishment of a level of expression of the transgene of interest or of the activity of the transcript of interest, then results therefrom.

The regulatory sequences comprising the repeat sequences of tetO are advantageously integrated within a tissue-specific amplifier/promoter, or can be used as a replacement for certain amplifying sequences (Rose et al., J. Biol. Chem., 272 (1997) 4735-4739; Agha-Mohammadi et al., Gene Ther, 5 (1998) 76-84). This system thus confers not only temporal targeting of the regulation of the transgene of interest, but also spatial targeting.

In one embodiment of the present invention, the coding sequence for the tTA transactivator and the TrRS promoter driving the transcription of the inhibitory transcript are carried on a single nucleic acid molecule. The latter can comprise, for example, the sequence encoding tTA under the control of a viral or tissue-specific promoter, then the tetracycline-repressible promoter (TrRS) cassette functionally linked with the sequence encoding the inhibitory transcript (O'Brien et al., Gene, 184 (1997) 115-120).

An alternative organization of bicistronic type comprising the TrRS expression cassette functionally linked to the sequence encoding an inhibitory transcript, followed by an IRES (Internal Ribosome Entry Site) sequence and by a coding sequence for the tTA, or vice versa, can also be used. Yet another example of organization comprises a bidirectional promoter which drives the expression of the tTA is of the inhibitory transcript. In the absence of tetracycline, the tTA is expressed and activates the transcription of the inhibitory transgene into an inhibitory transcript, which in turn inhibits the useful transcript or transcript of interest (Liang et al., Gene Ther., 3 (1996) 350-356).

The external repressor agent used according to this first embodiment can be tetracycline or one of the analogues thereof, such as doxycycline, anhydrotetracycline or oxytetracycline (Agha-Mohammadi et al., Gene Ther, 4 (1997) 993-997), capable of causing inhibition of the transcription of the inhibitory transgene, and therefore of the activity of the inhibitory transcript. The administration of tetracycline or of one of the analogues thereof makes it possible to lift the inhibition by the inhibitory transcript and thus to re-establish a biologically effective level of the transcript of interest. The level of expression of the transcript of interest can be advantageously correlated with the amount of tetracycline or of the analogue administered, in so far as the pharmacokinetic and pharmacodynamic properties of tetracycline and of the analogues thereof are well known to a person skilled in the art, and are, inter alia, detailed in the Vidal, and in the chapter “Antimicrobial Agents: Tetracyclines” in: Goodman and Gilman's The Pharmacological Basis of Therapeutics, 9th Edition, Joel G. Hardman, Alfred Goodman Gilman, Lee E. Limbird Eds.

Moreover, because of the high affinity of tetracycline for the tetR protein, tetracycline or one of its analogues can be used at low concentrations, and consequently, the side effects are minimal.

In one embodiment of the present invention, the sequence of the inhibitory transgene is placed under the control of a minimal promoter derived from the promoter of the thymidine kinase (TK) gene, or of the human CMV gene, upstream of which is a regulatory sequence as described, for example, in WO 96/30512.

The inhibition of the inhibitory transcript can also be obtained by inserting, into its sequence or its 5′ or 3′ ends, specific sequences such as the aptamers which are described in European application EP 99402552, and by Werstuck et al. (Science, 282 (1998) 296-298), and which have autocatalytic activity, for example, in the presence of a ligand. Thus, through insertion of an aptamer sequence, the inhibitory transcript acquires autocatalytic activity which can be activated in the presence of a specific ligand when reestablishment of transcript of interest activity is desired. The nucleotide sequence of the aptamer which is used to inhibit the inhibitory transcript can be any sequence encoding an RNA which has ligand-dependent autocatalytic activity. It involves, for example, hammerhead ribozymes, hepatitis delta virus ribozymes, Neurospora VS ribozymes, pinhead ribozymes, group I and II introns and RNAse P, or any artificially obtained functional derived sequence (Clouet-d'Orval et al., Biochemistry, 34 (1995) 11186-11190; Olive et al., EMBO J 14 (1995) 3247-3251; Rogers et al., J. Mol Biol, 259 (1996) 916-915). The size of the aptamer sequence may vary depending on its nature and its origin, but it may range between 20 and 200 bp. The location of the insertion of the aptamer sequences is generally determined using biocomputing software packages such as “RNA fold”, in order to ensure optimal stability and cleavage activity as a function of the environment and of the confirmation (Zuker M, Method Mol Biol, 25 (1994) 267-94; Stage-Zimmermann T K, RNA, 4 (1998) 875-889).

The inhibition of the inhibitory transcript can finally be carried out via a transacting ribozyme which, due to its sequence specificity for a portion of the inhibitory transcript, is capable of recognizing and of hybridizing with the inhibitory transcript, and thus of degrading it. In another embodiment of the present invention, the trans ribozyme is in the form of an allosteric ribozyme, i.e. it has ligand-dependent catalytic activity, which is, for example, activated in the presence of a ligand. Such allosteric ribozymes are well known to a person skilled in the art and are, for example, described by Soukup et al., Structure, 7 (1999) 783-791 and in WO 94/13791.

The activator ligands used are, for example, nucleic acids, proteins, polysaccharides or sugars, or alternatively any organic or inorganic molecules capable of binding to the aptamer sequence of the inhibitory transcript, or to a sequence of the allosteric ribozyme, by a molecular recognition mechanism, and thus of activating the catalytic activity (Famulok M, Curr Opin Struc Biol, 9 (1999) 324-329). These ligands are well known to a person skilled in the art and are, for example, described, inter alia, by Cowan et al. (Nucleic Acids Res., 28 (15) (2000) 2935-2942) and by Werstuck et al. (Science, 282 (1998), 296-298). By way of examples, mention may be made of antibiotics, such as doxycycline, pefloxacin, tobramycin or kanamycin, dyes such as the Hoechst dyes H33258 and H33342, mononucleotides such as FMN (flavin mononucleotide), ATP or cAMP, drugs such as theophylline, adjuvants and substitutes.

According to this embodiment, the transgene of interest is placed under the control of a constitutive promoter which is functional in the target tissue or cells of mammals and, for example, humans. Accordingly, the constitutive promoter driving the expression of the transcript of interest is, for example, tissue-specific.

According to a second embodiment of the present invention, the transcript of interest is activated, whereas the activity of the inhibitory transcript is either kept constant or inhibited concomitantly with the activation of the transcript of interest, in order to re-establish a sufficient level of expression or of biological activity of the latter.

The activation of the transcript of interest can be obtained by placing the sequence of the transgene of interest encoding the transcript of interest under the control of an inducible promoter. The transcript of interest can also be activated by acting on the stability of the latter.

The activity of the inhibitory transcript can then be kept constant, and in this case, the inhibitory transgene is placed under the control of a constitutive promoter and is not subjected to any inhibition via an aptamer or a ribozyme with ligand-dependent cis or trans catalytic activity.

According to one embodiment, the activity of the inhibitory transcript is repressed, as described above, concomitantly with the activation of the transcript of interest.

The constitutive or inducible promoters used in these embodiments are well known to a person skilled in the art. They can thus be any promoter or derived sequence of different, heterologous or homologous origin, which may or may not be tissue-specific, which is strong or weak, and which is functional in the target tissue or cells and thus capable of directing the transcription of a functionally linked sequence.

Mention may be made of promoter sequences of eukaryotic or viral genes. Among eukaryotic promoters, use may be made, for example, of ubiquitous promoters (promoter of the HPRT, phosphoglycerate kinase (PGK), α-actin, tubulin and histone genes), intermediate filament promoters (promoter of the GFAP, desmin, vimentin, neurofilament, keratin, etc. genes), therapeutic gene promoters (for example the promoter of the MDR, CFTR, Factor VIII and IX, ApoAI, ApoAII, albumin, thymidine kinase, etc. genes), tissue-specific promoters (promoter of the pyruvate kinase, villin, fatty acid-binding intestinal protein and smooth muscle α-actin gene, promoters specific for endothelial cells, such as the von Willebrand factor promoter, promoters specific for cells of myeloid and hematopoietic lines, such as the IgG promoter, the neuronal specific enolase promoter (Forss-Petter et al., Neuron, 5 (1990) 187); etc.), the promoter generating the V1 form of the mRNA of VAChT (acetylcholine transporter; Cervini et al., J. Biol. Chem., 270 (1995) 24654), promoters which are functional in a hyperproliferative cell (cancerous, restenosis, etc.), such as the promoter of the p53 gene, the promoter of the transferrin receptor, or alternatively promoters which respond to a stimulus (steroid hormone receptor, retinoic acid receptor, etc.) In the case of the latter, the external agents are specific transcriptional activating factors capable of binding in trans, either directly or via nuclear receptors, to a response element (RE) of the inducible promoter which directs the expression of the transcript of interest.

The rapamycin-mediated regulation system (PRS) (Rivera et al., Nat. Med., 2 (1996) 1028-1032) can also be used. It uses a two-part transcription factor comprising two chimeric peptides of human origin namely a DNA-binding ZFHD1-FKBP12 first chimeric protein and a second chimeric protein which results from the fusion of the truncated FRAP cellular protein and of a 189-amino acid C-terminal sequence of the NF-kB65 protein. In the presence of rapamycin, the ZFHD1-FKBP12 protein binds to the FRAP-p65 chimeric protein which activates the ZFHD1 dependent promoter. In an embodiment of the present invention, inert analogues of rapamycin, which can be administered for example orally or intravenously, are used as external activating agents for the activation of the promoter (Ye et al., Science, 283 (1999) 88-91).

In another embodiment of the present invention, the inducible promoter sequence for the transgene of interest is as described in French application FR 9907957, or by Frohnert et al. (J Biol. Chem., 274(1999)3970-3977), and comprises one or more response elements (PPREs) linked to a minimum transcriptional promoter. This system for activating the expression of the transgene of interest functions with PPAR α or γ (Peroxisome Proliferator Activated Receptor) nuclear receptors as transcriptional regulators. Advantageously, retinoid X receptors (RXRs), such as human RXRα, which are capable of heterodimerizing with PPARs and thus of synergizing the activation of the transgene of interest, are used as transcriptional coregulators (Mangelsdorf et al., Nature, 345 (1990) 224-229; Mangelsdorf et al., Genes Dev, 6 (1992) 329-344; Mangelsdorf et al., Cell, 83 (1995) 841-851; Wilson et al., Curr Op Chem Biol, 1 (1997) 235-241; Schulman et al., Mol and Cell Biol, 18 (1998) 3483-3494; Mukherjee et al., Arterioscler Thromb Vasc Biol, 18 (1998) 272-276). It is also possible to use a PPAR α or γ in its native form, without any modification of the primary structure, or a modified PPAR comprising one or more ligand binding sites or E/F domains, such as between 2 to 4 (Schoonjans et al., Biochim Biophys Acta, 1302 (1996) 93-109). The limits of the E/F domains vary from one PPAR to the other. By way of example, for the human PPARγ2 isoform, the E/F domain stretches from amino acid 284 to amino acid 505. Use is made advantageously, as a transcriptional regulator of the expression in vivo of the transgene of interest, of PPAR_{γ2γ2}, i.e. a modified human PPAR γ comprising two repeat domains E and F, the complete protein sequence of which is represented in the sequence SEQ ID NO: 1.

| | |
|---|---|
| MGETLGDSPIDPESDSFTDTLSANISQEMTMVDTEMPFWPTNFGISSVDLSVMEDESHSFDI | SEQ ID NO: 1 |
| KPFTTVDFSSISTPHYEDIPFTRTDPVVADYKYDLKLQEYQSAIKVEPASPPYYSEKTQLYN | |
| KPHEEPSNSLMAIECRVCGDKASGFHYGVHACEGCKGFFRRTIRLKLIYDRCDLNCRIHKKS | |
| RNKCQYCRFQKCLAVGMSHNAIRFGRMPQAEKEKLLAEISSDIDQLNPESADLRALAXHLYD | |
| SYIKSFPLTKAKARAILTGKTTDKSPFVIYDMNSLMMGEDKIKPKHITPLQEQSKEVAIRIF | |
| QGCQFRSVEAVQEITEYAKSIPGFVNLDLNDQVTLLKYGVHEIIYTMLASU4NKDGVMSEG | |
| QGFMTREFLKSLRKPFGDFMEPKFEFAVKFNALELDDSDLAIFIAVIILSGDRPGLLNVKPI | |
| EDIQDNLLQALELQLKLNHPESSQLFAIQJLQKMTDLRQIVTEHVQLLQVIKKTETDMSLHPL | |
| LQEIYKDLYAWAILTGKTTDKSPFVIYDMNSU4MGEDKIKFKHITPLQEQSKEVAIRIFQGC | |
| QFRSVEAVQEITEYAXSIPGFVNLDLNDQVTLLKYGVHEIIYTMLASLMNKDGVLISEGQGF | |
| MTREFLKSLRKPFGDFMEPKFEFAVKFNALELDDSDLAIFIAVIILSGDRPGLLNVKPIEDI | |
| QDNLLQALELQLKLNHPESSQLFAKLLQKMTDLRQIVTEHVQLLQVIKKTETDMSLHPLLQE | |
| IYKDLY | |

Moreover, the PPAR response element (PPRE), which is therefore a nucleic acid region capable of binding a PPAR and thus mediating a signal for activating transcription of the transgene of interest, can comprise one or more PPAR binding sites. Such sites are described in the prior art, for instance in various human promoters for example, such as the promoter of the human apolipoprotein All (Apoll) gene (Vu-Dac et al., J Clin Invest, 96(2), (1995), 741-750). It is also possible to use artificially constructed sites corresponding, for example, to the J region of the human ApoAll promoter located, for example, at nucleotides -734 to -716, with respect to the +1 transcription initiation point, of sequence TCAACCTTTACCCTGGTAG (SEQ ID NO: 2) or any other functional variant of this sequence. A sequence corresponding to the DR1 consensus region of sequence AGGTCAAAGGTCA (SEQ ID NO: 3) can also be used as a PPAR binding site.

PPARα-activating ligands, for example fibrates such as fibric acid and the analogues thereof, are used as external activator agents. As analogues of fibrin acid, mention may be made, for example, of gemfibrozyl (Atherosclerosis, 114(1) (1995) 61), bezafibrate (Hepatology, 21 (1995) 1025), ciprofibrate (BCE&M 9(4) (1995) 825), clofibrate (Drug Safety, 11 (1994) 301), fenofibrate (Fenofibrate Monograph, Oxford Clinical Communications, 1995), clinofibrate (Kidney International 44(6) (1993) 1352), pirinixic acid (Wy-14,643) or 5, 8, 11, 14-eicosatetraynoic acid (ETYA). These various compounds are compatible with biological and/or pharmacological use in vivo.

The external activator agents can also be chosen from natural and synthetic PPARγ ligands. As natural ligands, mention may be made of fatty acids and eicosanoids, such as for example linoleic acid, linolenic acid, 9-HODE or 5-HODE, and as synthetic ligands, mention may be made of thiazolidinediones, such as, for example, rosiglitazone (BRL49653), pioglitazone or troglitazone (see for example Krey G. et al., Mol. Endocrinol., 11 (1997) 779-791 or Kliewer S. and Willson T., Curr. Opin. in Gen. Dev., 8 (1998) 576-581) or the compound RG12525.

Similarly, it may involve promoter sequences derived from the genome of a virus, such as for example the promoters of the adenovirus genes ElA and MLP, the CMV early promoter, or alternatively the promoter of the RSV or MMTV LTR, the promoter of the herpesvirus TK gene, etc. In addition, these promoter regions can be modified by adding or deleting sequences.

Unlike known inducible systems, which have periods of deinduction of the exogenous gene, i.e. of return of the expression to a basic level, which are quite long due to the life span and/or to the difficulty of diffusion of the induction factors, the system according to the present invention ensures faster and more effective activation and consecutive inhibition of the exogenous gene. Specifically, the method according to the present invention makes it possible, simultaneously with the deinduction of the useful transcript, to lift the inhibition of the inhibitory transcript and thus to decrease, more rapidly and to a greatly lowered residual level, the expression of a transgene of interest.

According to one embodiment of the present invention, the inhibitory transcript is in the form of an antisense RNA, and is termed “inhibitory transcript of antisense RNA type”. The latter generally comprises a nucleotide sequence which is complementary to at least one portion of the transcript of interest and hybridizes selectively to the transcripts of interest via conventional Watson-Crick-type interactions. Generally, hybridization between at least two complementary nucleotide sequences is also referred to herein as a “Watson and Crick-type linkage”. The inhibitory transcript of antisense RNA type can therefore bind to the transcript of interest and, for example, block access to the cellular translation machinery at the 5′ end of the transcript of interest, when the latter is an mRNA, impede its translation into a protein and allow the suppression of the expression of the transgene of interest in vivo (Kumar et al., Microbiol. Mol. Biol., Rev, 62 (1993) 1415-1434). Such polynucleotides have, for example, been described in patents EP 92574 and EP 140308.

When the inhibitory transcript is of antisense RNA type, it can cover all or part of the coding sequence of the transcript of interest of mRNA type, or all or part of the 3′ or 5′ noncoding sequence. In another embodiment of the present invention, the antisense inhibitory transcript is complementary to the ribozyme-binding and translation initiation sequence (Coleman J et al., Nature, 315 (1990) 601-603). In yet another embodiment of the present invention, the inhibitory transcript is at least 10 ribonucleotides long.

The determination of the length and of the sequence of the nucleic acid encoding the inhibitory transcript can be carried out through a routine experiment comprising coinjecting and coexpressing the nucleic acids encoding the inhibitory transcript and the transcript of interest, and in verifying effective inhibition using diverse detection techniques known to a person skilled in the art, namely for example RT-PCR and diverse techniques for assaying the protein of interest and for detection on Western blot.

The nucleic acids encoding the transcript of interest and the inhibitory transcript of antisense type comprise advantageously the signals which allow transcription to be stopped and signals which allow its stabilization, such as for example a 5′ cap and a 3′ polyadenylation site, and optionally an intron.

According to this embodiment of the present invention, the inhibitory transcripts of antisense RNA type, which are coexpressed with the transgene of interest in a target tissue or target cells, are thus capable of effectively blocking the expression of the transgene of interest at the translational level, or the biological activity of the transcript of interest at the level of the target tissue or cells.

According to another embodiment of the present invention, the inhibitory transcript can also be in the form of a catalytic RNA or ribozyme which has the transcript of interest as its target, and is designated inhibitory transcript of ribozyme type. The ribozyme can be, for example, a cis ribozyme, i.e. act at the intracellular level in cis (Cech TR, Biosci Rep, 10(3) (1990), 239-261). In yet another embodiment of the present invention, it is a trans ribozyme, i.e. capable of degrading several transcripts of interest in trans (Robertson et al., Nature, 344 (1990) 467; Ellington et al., Nature, 346 (1990) 818; Piccirilli et al., Science, 256 (1992) 1420; Noller et al., Science, 256 (1992), 1416; Ellington et al., Nature, 355 (1992) 850; Bock et al. 355 (1992) 564; Beaudry et al., Science, 257 (1992) 635).

The inhibitory transcript of ribozyme type generally has two distinct regions. A first region exhibits a certain specificity for the transcript of interest and is therefore capable of binding to the latter, whereas the second region confers on the ribozyme its catalytic activity of cleaving, ligating and splicing the transcript of interest. Diverse types of ribozyme can be used, such as, for example, hammerhead ribozymes or circular ribozymes, hairpin ribozymes, lasso ribozymes, tetrahymena ribozymes or RNAse P (Clouet-d'Orval B. et al., Biochemistry, 34 (1995) 11186-90; Olive J. E. et al., EMBO J, 14 (1995) 3247-51; Rogers et al., J Mol Biol, 259 (1996), 916-25).

In one embodiment of the present invention, the inhibitory transcript of ribozyme type is allosteric, i.e. its catalytic activity is regulated by a ligand (Szostak, TIBS, 10 (1992) 89). Some allosteric ribozymes have spontaneous target RNA-cleaving activity, whereas others are activated or inhibited subsequent to a change in conformation or subsequent to a hybridization reaction. Other allosteric ribozymes, termed aptazymes, are endowed with ligand-dependent self-cleaving activity which is, for example, activated by the binding of a ligand. Such regulatable ribozymes which are described, inter alia, in International applications WO 94/13791 and WO 96/21730, and generally have a ribozyme sequence and a ligand binding sequence which ensures control of the cleavage activity. The inhibitory transcript of ribozyme type used in the present invention is, for example, inactivated by the binding of a ligand, i.e. it exerts constitutive catalytic activity against the transcript of interest in the absence of ligand, and can be inactivated by administering a ligand, in order to re-establish a biologically sufficient level of the transcript of interest (Forter et al., Science, 249 (1990) 783-786).

The size of the ribozyme inhibitory transcript can vary depending on its nature and/or its origin. It is generally between 10 and 500 base pairs, and may be less than 300 base pairs. The nucleic acid encoding the inhibitory transcript of ribozyme type can, for example, originate from RNA sequences of natural origin or be obtained by chemical synthesis for example using an automatic synthesizer.

The ligands used for regulating the allosteric ribozymes are, for example, nucleic acids, proteins, polysaccharides or sugars, or alternatively any organic or inorganic molecules capable of binding to the ribozyme inhibitory transcript and of inhibiting the cleavage reaction for the transcript of interest, or of binding to the aptazyme inhibitory transcript and thus of activating the self-cleaving reaction. In an embodiment of the present invention, the ligand is an external agent, such as a nontoxic agent or drug, which can be administered in vivo via diverse external routes, and thus act on the target cell or tissue in order to inhibit the allosteric ribozyme and to restore a sufficient concentration and activity of the transcript of interest. For example, the ligand may be an antibiotic, such as tetracycline, doxycycline or pefloxacin, or an adjuvant which is harmless for the organism to which it is administered.

According to this embodiment of the present invention, the inhibitory transcripts of ribozyme type, which are coexpressed with the transgene of interest in a target tissue or target cells, are thus capable of effectively blocking the expression of the transgene of interest at the translational level, or of decreasing the concentration of the transcript of interest by nuclease-, transferase- and polymerase-type enzymatic degradation, the biological activity of the transcript of interest at the level of the target tissue or cells, or alternatively its interaction with the cellular components.

Again according to another embodiment of the present invention, the inhibitor transcript is in the form of an RNA which forms triple helices and which is capable of associating with the transgene of interest or transcript of interest with which it is coexpressed in vivo. Such an RNA is described, inter alia, in application WO 95/18223, by Giovannangeli et al., (J. Am. Chem. Soc., 113(1991) (7775-7) and by Hélène et al. (CibaFound Symp., 209 (1997), 84-102), and encodes, for example, composite RNAs comprising at least:

a first region capable of forming a double helix with the single-stranded nucleic acid targeted at the level of the sequence of the transgene of interest, or with a portion of it,

a second region capable of forming a triple helix with the double helix thus formed, or with a portion of it, and

one or two arms linking the two regions, each of the regions possibly being continuous or discontinuous.

The polynucleotide according to this embodiment, for example,is generally more than 10 bases in length, and can be more than 15 bases. This length is adjusted by a person skilled in the art as a function of the length of the nucleic acid of the transgene of interest targeted which is single-stranded or of the transcript of interest, so as to ensure the stability, specificity and selectivity of the triple helix inhibitory transcript.

As described above, the method according to the present invention allows the transfer of foreign or exogenous genes and the control of their expression in an effective and reversible manner. This is advantageous when the therapeutic product of the transgene of interest has optimum action within a certain well defined concentration range and becomes toxic outside this concentration range (Dranoff et al. Proc. Natl. Acad. Sci., (1993) 3539-3543; Schmidt et al., Mol. Med. Today, 2 (1996) 343-348). Moreover, some clinical applications require a precise regulation of the expression of the transgene of interest at predefined biological or therapeutic levels, for the purpose of optimizing its activity in vivo.

In addition, the method for reversible negative regulation according to the present invention is useful, for example, when the expression of a transgene of interest, or the activity of the transcript of interest, must be maintained at its minimum, or even extinguished, over long periods of time and rapid induction is required at precise moments, whether for therapeutic or experimental needs.

The method for controlling the expression of an exogenous gene by reversible inhibition according to the invention makes it possible to control the expression of any transgene which has an experimental value and for which it is desired to study the function in vivo, or the involvement in molecular mechanisms or in cell signalling, such as for example receptors, transcription factors, transporters, etc., or of any transgene of interest encoding, for example, a product of therapeutic interest, whether it is a peptide, polypeptide, protein, ribonucleic acid, etc. In other embodiments of the present invention, the transgene of interest is a DNA sequence (cDNA, gDNA, synthetic, human, animal, plant, etc. DNA) encoding a protein product.

The transcript of interest can be an antisense sequence, the expression of which in the target cell makes it possible to control cellular mRNA transcription or gene expression. Such sequences can, for example, be transcribed, in the target cell, into RNAs which are complementary to cellular mRNAs, and thus block their translation into protein, according to the technique described in patent EP 140 308. The transcript of interest can also be a ligand RNA (WO 91/19813).

The present invention is, for example, suitable for expressing sequences encoding toxic factors. They can be, for example, poisons for cells (diphtheria toxin, pseudomonas toxin, ricin A, etc.), a product which induces sensitivity to an external agent (suicide genes: thymidine kinase, cytosine deaminase, etc.) or genes capable in inducing cell death (Grb3-3) (WO 96/07981), anti-ras ScFv (WO 94/29446), etc.). This system is therefore generally suited to, for example, antitumor therapy strategies, for example for the expression of cytokines, interferons, TNF or TGF, the uncontrolled production of which can have very marked side effects.

This system is also generally suitable for gene therapy strategies, such as angiogenesis using a gene for a growth factor such as for example FGF or VEGF. It is suitable for controlling the expression of a hormone, such as erythropoietin, or of anticytokines, such as the soluble TNF-α receptor used for anti-inflammatory therapy purposes.

According to the method of the present invention, the combination of the nucleic acid comprising the sequence of the transgene of interest encoding the transcript of interest and of the nucleic acid comprising the sequence encoding the inhibitory transcript is transferred simultaneously into the target tissue or cell so as to allow their coexpression. Various physical or mechanical techniques exist for carrying out the transfer of these nucleic acids, such as for example injection, the ballistic technique, electroporation, electropermeabilization, electrotransfer, sonoporation, techniques using electrical fields, microwaves, heat, hydrostatic pressure, or any suitable combination of these techniques (Budker et al., J. Gen. Medicine, 2 (2000) (76-88). In one embodiment of the present invention, the nucleic acid combination is introduced by injection and electrotransfer, i.e. by the action of an electrical field. The electrotransfer technique is, for example, described in applications WO 99/01157 and WO 99/01158, and by Aihara et al., Nat. Biotechnol., 16 (9) (1998) 867-870; Mir et al., Proc. Natl. Acad. Sci., 96 (1999), 4262-4267; Rizzuto et al., Proc. Natl. Acad. Sci., 96 (1999) 6417-6422. The nucleic acid molecules whose transfer is desired can be administered, for example, directly into the tissue or topically or systemically, and then one or more electric pulses of an intensity, for example, of between 1 and 800 volts/cm, such as between 20 and 200 volts/cm, are applied.

Alternatively, the nucleic acid combination according to the present invention can be injected in the form of naked DNA according to the technique described in application WO 90/11092. It can also be administered in a form which is complexed with a chemical or biochemical agent. As a chemical or biochemical agent, mention may be made, for example, of lipofectamine, which associates with the DNA by forming vesicules called lipoplexes, and other polymers, such as DEAE-dextran (Pagano et al., J. Virol., 1 (1967) 891), polyamidoamine (PAMAM), polylysine, polyethyleneimine (PEI), polyvinylpyrrolidone (PVP), or polyvinyl alcohol (PVA), etc., or even viral proteins which associate to form virosomes (Schoen et al., Gen Ther, 6 (1999), 5424-5431), or molecules derived from viral proteins (Kichler et al., J Virol, 74 (2000) 5424-5431). Mention may also be made of cationic proteins such as histones (Kaneda et al., Science, 243 (1989) 375) and protamines. The nucleic acids can also be incorporated into lipids in crude form (Feigner et al., PNAS, 84 (1987) 7413), or alternatively be incorporated into a vector such as a liposome (Fraley et al., J. Biol. Chem., 255 (1980) 10431) or a nanoparticle. Liposomes are phospholipid vesicules comprising an internal aqueous phase in which the nucleic acids can be encapsulated. The synthesis of liposomes and their use for transferring nucleic acids is known in the prior art (WO 91/06309, WO 92/19752, WO 92/19730). Nanoparticles are particles of small size, generally less than 500 nm, which are capable of transporting or vectorizing an active principle (such as a nucleic acid) in cells or in the blood circulation. Nanoparticles can comprise polymers comprising mainly degradable units, such as polylactic acid, optionally copolymerized with polyethylene glycol. Other polymers which can be used in the production of nanoparticles have been described in the prior art (EP 275 796; EP 520 889).

Another aspect of the present invention relates to vectors which include a nucleic acid comprising the sequence of a transgene of interest encoding a transcript of interest or useful transcript, and a nucleic acid comprising the sequence of an inhibitory transgene encoding an inhibitory transcript specific for the transcript of interest. The nucleic acids can be carried by the same vector or by different vectors. When they are carried by the same vector, they may be carried on the same strand.

The use of such a vector makes it possible, in fact, to improve the efficiency of transfer into the target cells, and also to increase its stability in said cells, thereby making it possible to obtain a long-lasting therapeutic effect. Moreover, the use of vectors also makes it possible to target certain populations of cells in which the therapeutic molecules must be produced.

The vector used can be of diverse origins, provided that it is capable of transforming plant and animal cells, and for example human cells. It can equally be a nonviral vector, such as a plasmid, an episome, a cosmid or an artificial chromosome, or a viral vector. In one embodiment of the present invention, a viral vector is used which can be chosen from adenoviruses, retroviruses, adeno-associated viruses (AAVs), herpesvirus, cytomegalovirus, vaccinia virus, etc. It can also be a phage, an invasive bacterium or a parasite.

Vectors which are derived from adenoviruses, retroviruses or AAVs, and which incorporate heterologous nucleic acid sequences, have been described in the literature [Akli et al., Nature Genetics, 3 (1993) 224; Stratford-Perricaudet et al., Human Gene Therapy, 1 (1990) 241; EP 185 573; Levrero et al., Gene, 101 (1991) 195; Le Gal la Salle et al., Science, 259 (1993) 988; Roemer et Friedmann, Eur. J. Biochem., 208(1992)211; Dobson et al., Neuron, 5(1990)353; Chiocca et al., New Biol., 2 (1990) 739; Miyanohara et al., New Biol., 4 (1992) 238; WO 91/18088).

Advantageously, the recombinant virus according to the invention is a defective virus. The term “defective virus” means a virus which is incapable of replicating in the target cell. Generally, the genome of the defective viruses used in the context of the present invention is therefore devoid of at least the sequences required for the replication of said virus in the infected cell. These regions can be either removed (totally or partially), made nonfunctional, or substituted with other sequences, such aswith the sequence of the double-stranded nucleic acid of the invention. In another embodiment of the present invention, the defective virus conserves the sequences of its genome which are required for encapsidation of the viral particles.

The method according to an embodiment of the present invention uses vectors, such as viral vectors, comprising the nucleic acid sequences of a transgene of interest and of the specific inhibitory transgene, wherein the transgene of interest expresses a toxic molecule or toxic factor of interest. In this embodiment, the corresponding inhibitory transgene can prevent the expression of the toxic molecules or toxic factors in the viral production cells, thereby avoiding toxicity for the viral production cells. Furthermore, when this embodiment is administered to target cells or tissues, then advantageous expression of these toxic molecules in target cells can be accomplished by treating the target cells or tissues with a repressor agent. Accordingly, the repressor agent can repress the inhibitory activity of the specific inhibitory transgene, thereby allowing the transgene of interest to express the toxic molecules or toxic factors of interest in the specific target cells or tissues.

The invention can be used for regulating the expression of a transgene of interest in various types of cell, tissue or organ, in vivo. For example, it can be a cell, a tissue or an organ of plant or animal origin, such as of mammalian origin, and for example of human origin. By way of illustration, mention may be made of muscle cells (or a muscle), hepatic cells (or the liver), cardiac cells (or the heart, the arterial or vascular wall), nerve cells (or the brain, the medulla, etc.) or tumor cells (or a tumor). In one embodiment of the present invention, the compositions, constructs and method according to the invention are used for the regulated expression of a transgene of interest in a muscle cell or a muscle in vivo. The results given in the examples illustrate generally the advantages of the invention in vivo in this type of cell.

Another aspect of the present invention relates to cells or tissues of animal or plant origin which can be obtained by the method as described above, and which comprise a nucleic acid comprising the sequence of a transgene of interest encoding a transcript of interest, and a nucleic acid comprising the sequence of an inhibitory transgene encoding an inhibitory transcript specific for the transcript of interest. The tissues according to the present invention are, for example, tissues of animal or plant origin which are reconstituted ex vivo, to give for example organoids or neo-organoids, the cells of which have been modified so as to express the biological product of the transgene of interest according to the control method of the present invention, and which can thus be reimplanted (Vandenburgh et al., Hum. Gen Ther., 9(17) (1998) 2555-2564; Powell et al., Hum Gen Ther, 10(4), (1999) 565-577; MacColl et al., J. Endocrinol, 162(1) (1999) 1-9).

Yet another aspect of the present invention relates to a composition which can be administered in vivo, comprising the nucleic acid sequence of a transgene of interest encoding a transcript of interest or useful transcript, the nucleic acid sequence of an inhibitory transgene encoding an inhibitory transcript specific for the transcript of interest, and a suitable vehicle.

The present invention also relates to a composition which can be administered in vivo, comprising at least one vector which includes the nucleic acid sequence of a transgene of interest encoding a transcript of interest or useful transcript, the nucleic acid sequence of an inhibitory transgene encoding an inhibitory transcript specific for said transcript of interest, and a suitable vehicle, the transcripts of interest and inhibitory transcripts possibly being activated or inhibited with an external agent.

The present invention also relates to a pharmaceutical composition intended to be administered in vivo, comprising at least one vector which includes the nucleic acid sequence of a transgene of interest encoding a transcript of interest or useful transcript, and of a nucleic acid encoding an inhibitory transcript specific for said transcript of interest, and a suitable vehicle, the transcripts of interest and inhibitory transcripts possibly being activated or inhibited with an external agent.

The present invention also relates to a medicinal product comprising at least one vector which includes the nucleic acid sequence of a transgene of interest encoding a transcript of interest or useful transcript, the nucleic acid sequence of an inhibitory transgene encoding an inhibitory transcript specific for said transcript of interest, and a suitable vehicle, the transcript of interest and inhibitory transcripts possibly being activated or inhibited with an external agent.

According to the present invention, any vehicle suitable for topical, cutaneous, oral, vaginal, parenteral, intranasal, intravenous, intramuscular, subcutaneous, intraocular, transdermal, etc. administration, for example, is used.

In one embodiment of the present invention, a pharmaceutically acceptable vehicle is used for an injectable formulation, such as for direct injection into the desired organ, or for any other administration. It can involve, for example, sterile, isotonic solutions or dry, such as lyophilized, compositions, which, by adding, depending on the case, sterilized water or physiological saline, allow the preparation of injectable solutes. The concentrations of nucleic acids, comprising the sequences of the transgene of interest encoding a transcript of interest and of the inhibitory transgene encoding the inhibitory transcript, which are used for the injection, as well as the number of administrations and the volume of the injections, can be adjusted as a function of various parameters, and, for example,as a function of the method of administration used, of the pathology concerned or of the transgene of interest whose expression it is desired to regulate, or as a function of the desired duration of the treatment.

Among the transgenes of interest for the purpose of the present invention, mention may be made of the genes encoding

enzymes, such as α-1-antitrypsin, proteinases (metalloproteinases, urokinase, uPA, tPA and streptokinase), proteases which cleave precursors to liberate active products (ACE, ICE) or the antagonists thereof (TIMP-1, tissue plasminogen activator inhibitor PAI, TFPI);

blood derivatives such as the factors involved in clotting: factors VII, VIII and IX, complement factors, thrombin;

hormones, or the enzymes involved in the hormone synthetic pathway, or the factors involved in controlling the synthesis, the excretion or the secretion of hormones, such as insulin, insulin-like factors (IGFs) or growth hormone, ACTH, the enzymes for synthesizing sex hormones;

lymphokines and cytokines: interleukins, chemokines (CXC and CC), interferons, TNF, TGF, chemotactic factors or activators such as MIF, MAF, PAF, MCP-1, eotaxin, LIF, etc. (French patent FR 2 688 514);

growth factors, for example IGFs, EGFs, FGFs, KGFs, NGFs, PDGFs, PIGFs, HGFs, proliferins;

angiogenic factors such as VEGFs or FGFs, angiopoietin 1 or 2, endothelin;

the enzymes for synthesizing neurotransmitters;

trophic factors, for example neurotrophic factors for treating neurodegenerative diseases, traumas which have damaged the nervous system, or retinal degeneration, for instance members of the neurotrophin family, such as NGF, BDNF, NT3, NT4/5, NT6, the derivatives thereof and related genes—the members of the CNTF family, such as CNTF, axokine and LIF, and the derivatives thereof—IL6 and the derivatives thereof—cardiotrophin and the derivatives thereof—GDNF and the derivatives thereof—the members of the IGF family, such as IGF-1 or IFGF-2, and the derivatives thereof—the members of the FGF family, such as FGF 1, 2, 3, 4, 5, 6, 7, 8 or 9, and the derivatives thereof, TGFβ;

bone growth factors;

hematopoietic factors, such as erythropoietin, GM-CSFs, M-CSFs, LIFs, etc.;

proteins of the cellular architecture, such as dystrophin or a minidystrophin (French patent FR 2 681 786), suicide genes (thymidine kinase, cytosine deaminase, cytochrome P450 enzymes), the genes of hemoglobin of other protein transporters;

genes corresponding to the proteins involved in lipid metabolism, such as apolipoprotein chosen from the apolipoproteins A-I, A-II, A-IV, B, C-I, C-II, C-III, D, E, F, G, H, J and apo(a), metabolic enzymes, such as for example lipases, lipoprotein lipase, hepatic lipase, lecithin-cholesterol acyltransferase, cholesterol 7-alpha-hydroxylase or phosphatidyl acid phosphatase, or alternatively lipid transfer proteins, such as the transfer protein for cholesterol esters or the transfer protein for phospholipids, an HDL-binding protein or a receptor chosen for example from LDL receptors, chylomicron receptors and scavenger receptors, and leptin for the treatment of obesity;

factors which regulate blood pressure, such as the enzymes involved in NO metabolism, angiotensin, bradykinin, vasopressin, ACE, renin, the enzymes encoding the mechanisms of synthesis or of release of prostaglandins, of thromboxane, or of adenosine, adenosine receptors, kallikreins and kallistatins, ANP, ANF, diuretic or antidiuretic factors, the factors involved in the synthesis, metabolism or release of mediators such as histamine, serotonin, catecholamines or neuropeptides;

anti-angiogenic factors, such as the Tie-1 and Tie-2 ligand, angiostatin, the factor ATF, the derivatives of plasminogen, endothelin, thrombospondins 1 and 2, PF-4, interferon α or β, interleukin 12, TNFα, the urokinase receptor, flt1, KDR, PAI1, PAI2, TIMP1, the prolactin fragment;

factors which protect against apoptosis, such as the AKT family;

proteins which are capable of inducing cell death, which are either active in themselves, such as caspases, of the “prodrug” type requiring activation by other factors, or proteins which activate prodrugs into agents causing cell death, such as herpesvirus thymidine kinase or deaminases, and which allow, for example, anticancer therapies to be envisaged;

proteins involved in intercellular contacts and adhesion: VCAM, PECAM, ELAM, ICAM, integrins, catenins;

extracellular matrix proteins;

proteins involved in cell migration;

proteins of the signal transduction type, of the type FAK, MEKK, p38 kinase, tyrosine kinases, serin-threonine kinases;

proteins involved in cell cycle regulation (p21, p16, cylins) and dominant negative mutant or derived proteins which block the cell cycle and which can, where appropriate, induce apoptosis;

transcription factors: jun, fos, AP1, p53 and the proteins of the p53 signalling cascade;

cell structure proteins, such as intermediate filaments (vimentin, desmin, keratins), dystrophin or the proteins involved in muscle contractility and the control of muscle contractility, for example the proteins involved in calcium metabolism and calcium fluxes in cells (SERCA).

In the case of proteins which function via ligand and receptor systems, use of the ligand (for example FGF or VEGF) or the receptor (FGF-R, VEGF-R) is conceivable. Mention may also be made of genes encoding fragments or mutants of ligand or receptor proteins, such as of the abovementioned proteins, which have either greater activity than the whole protein, or antagonist activity, or even activity of the “dominant negative” type compared with the initial protein (for example, fragments of receptors which inhibit the availability of circulating proteins, possibly combined with sequences which induce secretion of these fragments compared with anchoring in the cell membrane, or other systems for modifying the intracellular trafficking of these ligand-receptor systems so as to divert the availability of one of the elements), or which even have their own particular activity which is different from that of the total protein (ex. ATF).

Among the transgenes of interest encoding proteins or peptides secreted by the tissue, mention should be made of antibodies, variable fragments of single chain antibodies (ScFvs), or any other antibody fragment which has recognition capabilities, for its use in immunotherapy, for example for the treatment of infectious diseases, of tumors or of autoimmune diseases such as multiple sclerosis (anti-idiotype antibodies), and ScFvs which bind to pro-inflammatory cytokines, such as for example IL1 and TNFα, for the treatment of rheumatoid arthritis. Other transgenes of interest used in the medicinal product according to the invention encode, in a nonlimiting way, soluble receptors, such as, for example, the soluble CD4 receptor or the soluble TNF receptor, for anti-HIV therapy, the TNFα receptor or the soluble IL1 receptor, for the treatment of rheumatoid arthritis, or the soluble acetylcholine receptor, for the treatment of myasthenia; substrate peptides or enzyme inhibitors, or peptides which are agonists or antagonists of receptors or of adhesion proteins, for instance for the treatment of asthma, of thrombosis of restenosis, of metastases or of inflammation, for example; artificial, chimeric or truncated proteins. Among hormones of fundamental interest, mention may be made of insulin in the case of diabetes, growth hormone and calcitonin. Mention may also be made of proteins capable of inducing antitumor immunity or stimulating the immune response (IL2, GM-CSF, IL12, etc.). Finally, mention may be made of cytokines which decrease the T_{H1} response, such as IL 10, IL4 or IL 13.

Other transgenes which are of value and can also be used in the compositions and medicinal products according to the present invention have been described, for example, by McKusick, V. A. (Mendelian Inheritance in man, catalogs of autosomal dominant, autosomal recessive, and X-linked phenotypes. Eighth edition. Johns Hopkins University Press (1988)), and in Stanbury, J. B. et al. (The metabolic basis of inherited disease, Fifth Edition. McGraw-Hill (1983)). The transgenes of interest cover the proteins involved in the metabolism of amino acids, of lipids and of other cell components.

Mention may thus be made, in a nonlimiting way, of genes associated with diseases of carbohydrate metabolism, such as for example fructose-1-phosphate aldolase, fructose-1,6-diphosphatase, glucose-6-phosphatase, lysosomal α-1,4-glucosidase, amylo-1,6-glucosidase, amylo-(1,4:1,6)-transglucosidase, muscle phosphorylase, muscle phosphofructokinase, phosphorylase-b kinase, galactose-1-phosphate uridyl transferase, all enzymes of the pyruvate dehydrogenase complex, pyruvate carboxylase, 2-oxoglutarate glyoxylase carboxylase or D-glycerate dehydrogenase.

Mention may also be made of:

genes associated with diseases of amino acid metabolism, such as, for example, phenylalanine hydroxylase, dihydrobiopterin synthetase, tyrosine aminotransferase, tyrosinase, histidinase, fumarylacetoacetase, glutathione synthetase, γ-glutamylcysteine synthetase, ornithine-δ-aminotransferase, carbamoyl phosphate synthetase, ornithine carbamoyltransferase, argininosuccinate synthetase, argininosuccinate lyase, arginase, L-lysine dehydrogenase, L-lysine -ketoglutarate reductase, valine transaminase, leucine-isoleucin transaminase, branched-chain 2-keto acid decarboxylase, isovaleryl-CoA dehydrogenase, acyl -CoA dehydrogengase, 3-hydroxy-3-methylglutaryl-CoA lyase, acetoacetyl-CoA 3-ketothiolase, propionyl-CoA carboxylase, methylmalonyl-CoA mutase, ATP: cobalamin adenosyltransferase, dihydrofolate reductase, methylenetetrahydrofolate reductase, cystathionine β-synthetase, the sarcosine dehydrogenase complex, proteins belonging to the glycine-cleaving system, β-alanine transaminase, serum carnosinase, brain homocarnosinase.

genes associated with diseases of fat and fatty acid metabolism, such as, for example, lipoprotein lipase, apolipoprotein C-II, apolipoprotein E, other apolipoproteins, lecithin-cholesterol acyltransferase, LDL receptor, liver sterol hydroxylase, “phytanic acid” α-hydroxylase.

genes associated with lysosomal deficiencies, such as, for example, lysosomal α-L-iduronidase, lysosomal iduronate sulfatase, lysosomal heparan N-sulfatase, lysosomal N-acetyl-α-D-glucosaminidase, acetyl-CoA: lysosomal α-glucosamine N-acetyltransferase, lysosomal N-acetyl-α-D-glucosamine-6-sulfatase, lysomal galactosamine-6-sulfate sulfatase, lysosomal β-galactosidase, lysosomal arylsulfatase B, lysosomal β-glucuronidase, N-acetylglucosaminylphosphotransferase, lysosomal α-D-mannosidase, lysosomal α-neuraminidase, lysosomal aspartylglycosaminidase, lysosomal α-L-fucosidase, lysosomal acid lipase, lysosomal acid ceramidase, lysosomal sphingomyelinase, lysosomal glucocerebrosidase and lysosomal galactocerebrosidase, lysosomal galactosylceramidase, lysosomal arylsulfatase A, α-galactosidase A, lysosomal acid β-galactosidase, lysosomal hexosaminidase A α-chain.

Mention may also be made, in a nonrestrictive way, of genes associated with diseases of steroid and lipid metabolism, genes associated with diseases of purine and pyrimidine metabolism, genes associated with diseases of porphyrin and heme metabolism, genes associated with diseases of the metabolism of connective tissue of and of bone, as well as genes associated with diseases of the blood and of the hematopoietic organs, of muscles (myopathy), of the nervous system (neurodegenerative diseases) or of the circulatory system (treatment of ischemias and of stenosis, for example) and genes involved in mitochondrial genetic diseases.

The present invention also relates to the use of the combination as described above, for preparing a medicinal product intended for treating certain genetic abnormalities or deficiencies, such as for example mitochondrial genetic diseases, hemophilia and β-thalassemia.

In addition, a subject of the invention is the use of the combination according to the invention for preparing a medicinal product intended for treating and/or for preventing certain diseases such as, for example, ischemia, stenosis, myopathies, neurodegenerative diseases, metabolic diseases such as lysosomal diseases, inflammatory diseases such as rheumatoid arthritis, hormonal disorders such as diabetes, cardiovascular diseases such as hypertension, or hyperlipidemias such as obesity.

A subject of the present invention is also the use of the combination as described above, for preparing an anticancer medicinal product, or for preparing vaccines, for example antitumor DNA.

Another aspect of the present invention relates to transgenic animals which express a transgene of interest encoding a transcript of interest, and an inhibitory transgene encoding an inhibitory transcript specific for the transcript of interest, in one or more cell types. The methods for generating transgenic animals, such as transgenic mice, are now well known to a person skilled in the art, and are, for example, described by Hogan et al. (1986) A Laboratory Manual, Cold Spring Harbor, New York, Cold Spring Harbor Laboratory.

According to the present invention, the nucleic acids described above are transferred into nonhuman fertilized oocytes by microinjection, while implanting the oocyte into a carrier female in order for it to develop. Generally, the nucleic acids are integrated into the genome of the cell from which the transgenic animal develops, and remain in the genome of the adult animal, such that expression of the transgene of interest and of the inhibitory transgene in one or more cells or tissues of the transgenic animal can be observed. The transgenic animals carrying the nucleic acid sequences of the transgene of interest and of the inhibitory transgene can also be crossed with other transgenic animals carrying other transgenes.

As transgenic animals thus produced, mention may be made for example of mice, goats, sheep, pigs, cows or any other domestic animal. Such transgenic animals have a phenotype which is similar to the wild-type animals, however the transgene or transcript of interest is restored when an external agent which is a repressor of the inhibitory transcript and/or of an agent which is an activator of the activity of the transcript of interest is administered to the animal.

These transgenic animals are used to simulate the physiopathology of certain human or animal diseases, and therefore constitute experimental models of human or animal diseases. For example, in a host animal, the transgene of interest likely to be involved in a pathology can be introduced with its specific inhibitory transgene, without causing the appearance of a particular phenotype. The expression of the transgene of interest studied can then be modulated by administrating an external agent which is a repressor of the inhibitory transcript, and/or an external agent which is an activator of the transcript of interest, in order to determine the relationship which exists between the expression of this gene and the appearance of a pathological phenotype. Such an approach has a general advantage over the conventional knock out technique, since the transgenic animals according to the present invention allow inactivation of a transgene of interest which is not only total, but also reversible. The approach also allows the possibility of regulating the expression of the transgene of interest more effectively.

A final aspect of the present invention relates to transgenic plants and plant cells comprising, in their genome, a nucleic acid comprising the sequence of a transgene of interest encoding a transcript of interest, and a nucleic acid comprising the sequence of an inhibitory transgene encoding an inhibitory transcript specific for the transcript of interest. These plants can be obtained by the conventional techniques of plant transgenesis. Plasmids carrying the nucleic acids encoding the transgene or transcript of interest and the inhibitory transcript, placed under the control of transcription promoters which are naturally functional in plants, are introduced, for example, into a strain of Agrobacterium tumefaciens. The transformation of the plants can then be carried out using standard transformation and regeneration protocols (Deblaere et al., Nucleic Acid Research, 13 (1985) 4777-4788; Dinant et al., European Journal of Plant Pathology, 104 (1998) 377-382).

Constitutive promoters, such as for example the 35S promoter of the cauliflower mosaic virus (CaMV) (Odell et al., Nature, 313 (1985) 810-812), can be used. To direct the expression of the transcript of interest, it is possible to use inducible promoters, such as glucocorticoid-inducible promoters which are activated, inter alia, by dexamethasone (Aoyama et al., Plant J., 11 (1997) 605-612; Aoyama et al., Gene Expression in Plants, (1999) 44-59), the ethanol-inducible system (Caddick et al., Nat Biotechnol, 16 (1998) 177-180), or systems of transcriptional activation by steroid hormones such as δ-estradiol (Bruce et al., Plant Cell, 12 (2000) 65-80). Alternatively, use is made of an ecdysone-inducible transcriptional system as described by Martinez et al. (Plant J, 19 (1999) 97-106), which functions with a hybrid activator comprising the glucocorticoid receptor (GR) and VP16 transactivation sequences, the GR DNA-binding domain and the ecdysone receptor hormone-dependent regulation domain. The latter system can be activated, inter alia, with a nonsteroidal ecdysone agonist, RH5992, and makes it possible, therefore, to restore a level of expression of the transgene of interest in the activated state. However, although this regulation system gives a high basal level in the nonactivated state, when it is used to drive the expression of a transgene of interest in coexpression with an inhibitory transcript for this transgene of interest, according to the present invention, the basal level of the transgene of interest is greatly lowered.

According to this latter aspect, a cytotoxic, or even lethal, foreign gene can be expressed in a limited manner over a short lapse of time, without inhibiting the regeneration of the plant transduced and while limiting cell death. This system for reversibly inhibiting the expression of the transgene of interest is, consequently, extremely useful for certain applications of plant production biotechnology, and in the context of fundamental agronomic research.

Thus, the present invention is generally useful for studying genes whose overexpression, or even basic expression, has deleterious effects for the organism in which they are expressed. By way of examples, an uncontrolled production of cytokines in a plant causes, for example, the appearance of abnormal phenotypes during development, such as the absence of roots, loss of the apical dominance, sterility, or cell toxicity which, in the case of plants, blocks the regeneration of plant tissues or even leads to problems of lethality.

The method for reversibly inhibiting, according to the present invention, exogenous genes may, moreover, prove useful for studying the stability of the product of the transgene of interest (Gil et al., EMBO J., 15 (1996), 1678-1686), or evaluating the turnover of the product of an exogenous gene.

The transgenic plants according to the present invention carrying the constructs of the transgene of interest encoding a transcript of interest and of the inhibitory transgene encoding an inhibitory transcript specific for the transcript of interest, according to the present invention, can also be used for studying certain molecular mechanisms and gene interactions. For example, when the expression of certain genes leads to cell death, the transgenic lines carrying both the sequences of the lethal transgene of interest and of its inhibitory transcript can be used to isolate the mutants which make it possible to subsequently study the molecular mechanisms and interactions of cell death. Besides circumventing the lethal phenotype, the system according to the present invention facilities the functional analysis of certain genes and of their involvement in the appearance of a phenotype, as well as their possible implications in certain signal transduction pathways.

Also, the method according to the invention makes it possible to facilitate the study of plant genes which are liable to affect the development of the plant at the early stages, but may play a role at later stages of development. The mutations of these genes affect the development of the plant and, consequently, prevent the study of the possible late functions of these genes. Plants transformed with the sequence carrying the transgene of interest and its inhibitory transcript can follow a normal early development, and the administration of a suitable external agent at a subsequent stage of development makes it possible advantageously to restore the expression of the genes in question and to determine their late functions. The plant chimeras according to the invention are therefore capable of providing novel information, for example on signalling mechanisms in plants.

The following examples are intended to illustrate the invention without limiting the scope thereof.

### EXAMPLES

### Example 1

Construction of the Plasmids Carrying the Cytomegalovirus (CMV) Early Amplifier/promoter

1.1 Plasmid pXL3031 (Luciferase Plasmid)

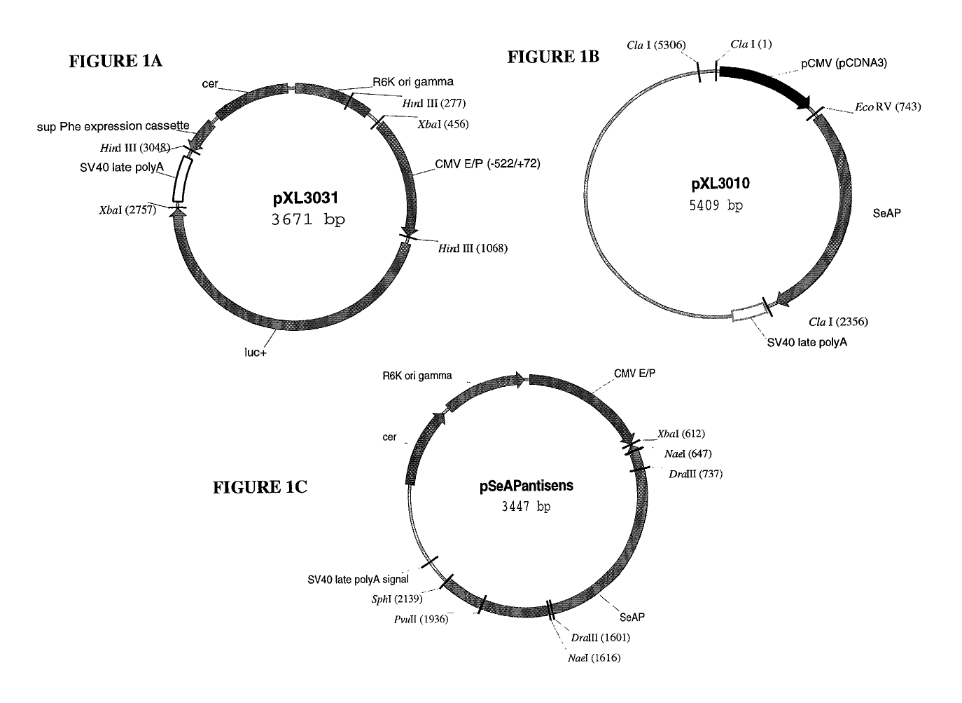

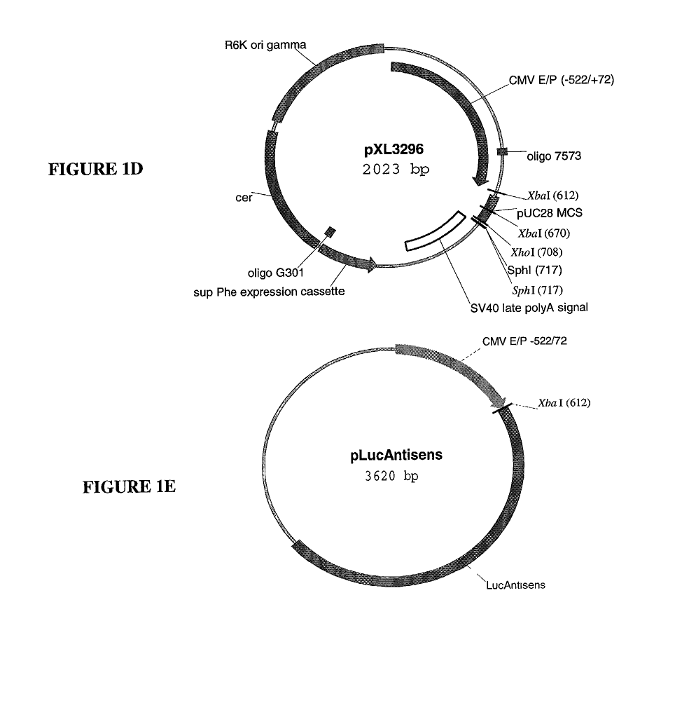

The plasmid pXL3031 is also a pCOR plasmid described in pCOR (Soubrier et al., Gen Ther, 6 (1999) 1482-1488), and comprised, for example, the luciferase reporter under the control of the CMV promoter. A schematic representation of this plasmid is given in FIG. 1A.

1.2 Plasmid pXL3010 (SeAP Plasmid)

The plasmid pXL3010 was constructed by ligating, into an MIuI/SaII fragment of pGL3-basic (Promega), an MIuI/SphI fragment of pCDNA3-basic (Invitrogen) comprising the human cytomegalovirus early promoter (hCMV-IE), the SeAP gene extracted from pSeAP-basic (Clontech) with SphI/CIaI and a CIaI/SaII fragment comprising the late polyadenylation signal of the simian virus (SV40 polyA) amplified from pGL3-basic by a polymerase chain reaction with the following primers (5′

| | |
|---|---|
| (5′-ATGCATCGATGGCCGCTTCGAGCAGACATG-3′ | (SEQ ID NO:4) and |
| 5′-ATGCGTCGACTCTAGCCGATTTTACCACATTTGTAGAGG-3′) | (SEQ ID NO:5). |

A schematic representation of this plasmid is given in FIG. 1B.

1.3 Plasmid pSeAPantisense (Plasmid Antisense SeAP in pCOR)

A DNA fragment comprising the SeAP gene was prepared by PCR using the plasmid pXL3010 as a matrix and oligonucleotides 1

| | |
|---|---|
| (5′ CGAGCATGCTGCTGCTGCTGCTGCTGCTGGGCC 3′) | (SEQ ID NO:6) and 2 |
| (5′ GGGTCTAGATTAACCCGGGTGCGCGGCGTCGGT 3′) | (SEQ ID NO:7) |

as primers. These oligonucleotides were located at positions 765-797 and 2290-2267, respectively, on the plasmid pXL3010.

This fragment was then digested with the XbaI and SphI restriction enzymes, purified on 0.8% agarose gel, extracted using the Jetsorb kit, and then cloned, in the antisense direction with respect to the CMV promoter, into the plasmid pXL3296, which had been digested beforehand with SphI and XbaI, so as to obtain the plasmid pSeAPantisense. A schematic representation of this plasmid is given in FIG. 1C.

1.4 Plasmid pXL3296 (Empty pCOR Plasmid)

The plasmid pXL3296 is a pCOR plasmid (Soubrier et al., Gen Ther, 6 (1999) 1482-1488) and includes the ORI γ of R6K, the expression cassette of the phenylalanine suppresser tRNA (sup Phe), and a −522/+72 portion of the early promoter/enhancer of the CMV virus. A schematic representation of this plasmid is given in FIG. 1D.

1.5 Plasmid pLucAntisense (Plasmid Antisense Luciferase in pCOR)

The plasmid pXL3031 was digested with HindIII and treated with the Klenow fragment in order to make the ends blunt. After ethanol precipitation, the fragment was digested with XbaI at 37° C. for 2 hours. After purification on 0.8% agarose gel, the approximately 1.6 kb fragment comprising the luciferase gene was extracted using the Jetsorb kit. The 1.6 Kb XbaI fragment of the luciferase gene was then cloned, in the antisense direction with respect to the CMV promoter, into the plasmid pXL3296, which beforehand had been digested with XhoI and treated with the Klenow fragment in the presence of deoxynucleotide triphosphates at 37° C. for 30 minutes in order to make the end blunt, so as to obtain the plasmid pLucAntisense. A schematic representation of this plasmid is given in FIG. 1E.

### Example 2

Construction of Plasmids Carrying the Tetracycline-repressible Promoter (TrRS)

2.1 Plasmid pTetLucAntisense (Plasmid Antisense Luciferase in pTet-Splice) and Plasmid pTetLuc (Plasmid Luciferase in pTet-Splice)

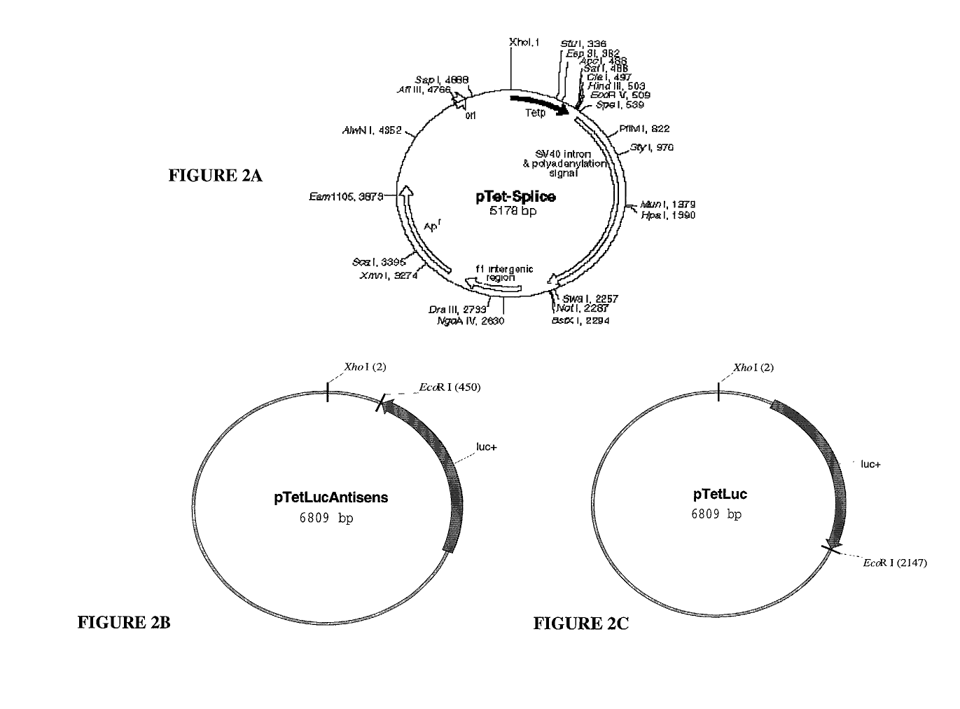

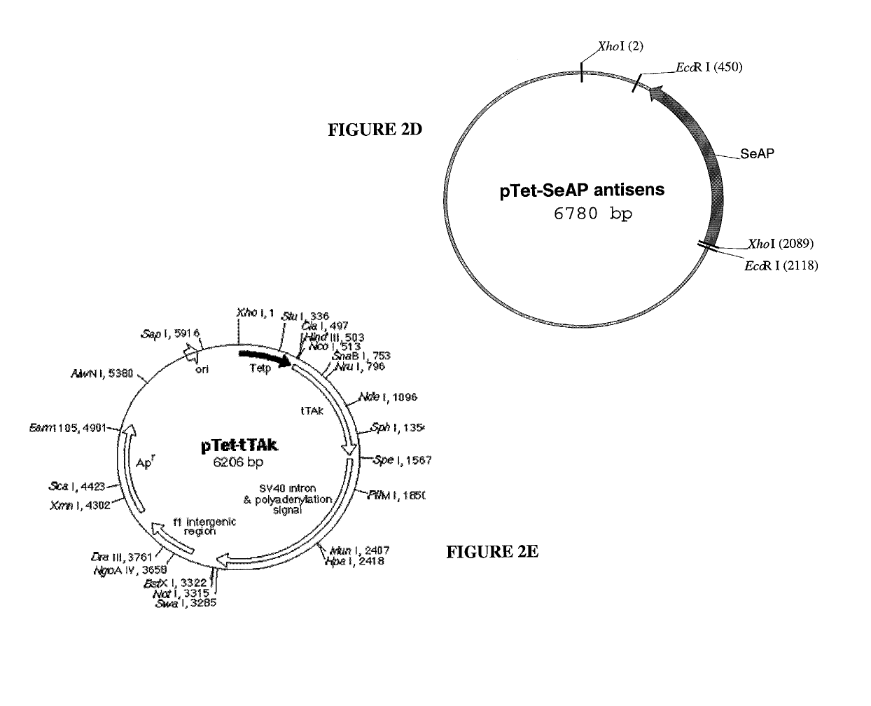

The approximately 1.7 kb HindIII and XbaI fragment comprising the luciferase gene was digested from the plasmid pXL3031,treated with the Klenow fragment so as to fill the ends and cloned, in the sense and antisense direction, into the plasmid pTetSplice (Gibco BRL; FIG. 2A), which beforehand had been digested with EcoRI, treated with the Klenow fragment and dephosphorylated, in order to obtain the plasmids pTetLuc and pTetLucAntisense, respectively. A schematic representation of these two plasmids is given in FIGS. 2C and 2B, respectively.

2.2 Plasmid pTetSeAPantisense (Plasmid Antisense SeAP in pTet-Splice)

The approximately 1.6 kb ClaI and EcoRV fragment comprising the SeAP gene was digested from the plasmid pXL3010 and cloned into the plasmid pTet-Splice, the map of which is given in FIG. 2A (Gibco BRL), which had been digested beforehand with ClaI and EcoRV, so as to give pTetSeAPantisense. A schematic representation of this plasmid is given in FIG. 2D.

2.3 Plasmid pTet-tTak

The fragment comprising the sequence of the transactivator tTA was obtained from the plasmid pUHD15-1 as described by Gossen et al. (Proc Natl Acad Sci., 89 (1992) 5547-5551) and cloned into the plasmid pTet-Splice (FIG. 2A) (Gibco BRL), which had been digested beforehand with HindIII and SpeI, so as to give pTet-tTAk. A schematic representation of this plasmid is given in FIG. 2E.

### Example 3

Construction of the Plasmids Comprising Shorter Fragments of the SeAPantisense Gene

3.1 Plasmid pGJA1 (Plasmid SeAPantisense 5′ End)

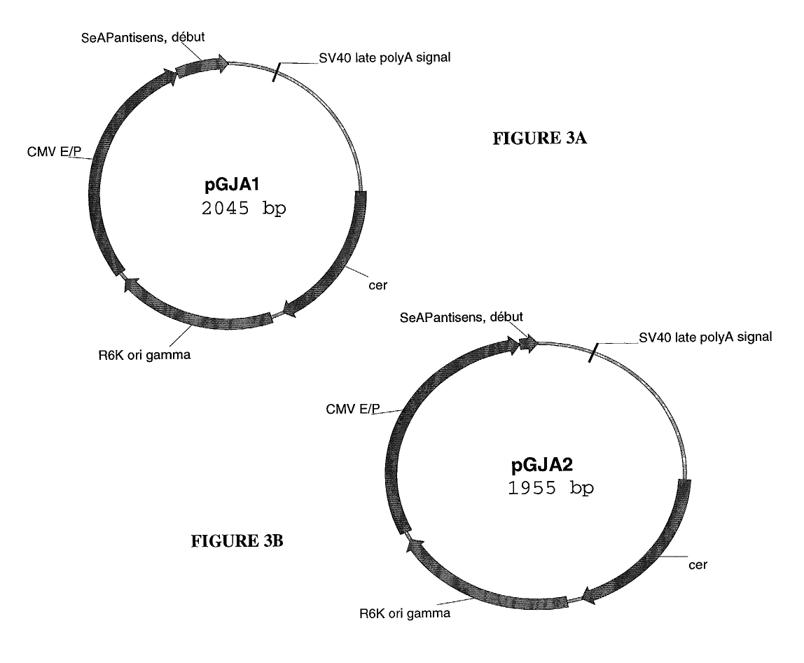

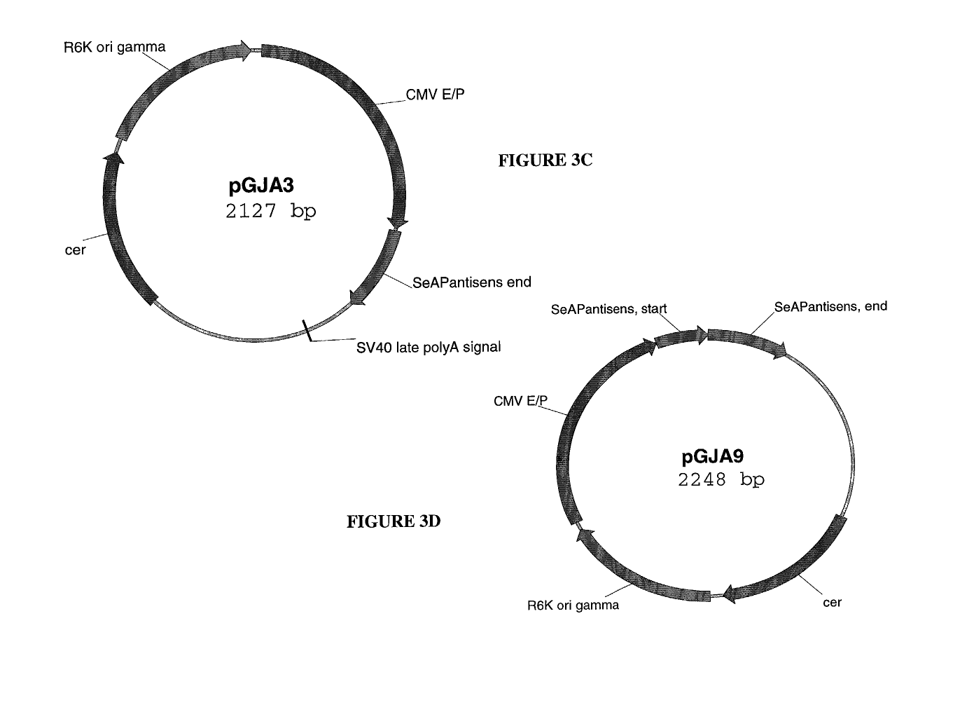

The plasmid pGJA1 was constructed by removing the major 5′ portion of the SeAPantisense gene from the plasmid pSeAPantisense (Example 1.3 and FIG. 1C) using the DraIII and Sph1 enzymes. The ends were ligated after treatment with the Klenow enzyme which makes the ends blunt. The fragment removed corresponded to the portion between positions 737 and 2139 of the SeAPantisense gene. The remaining portion comprised the first 125 bases (5′), and thus the end of the 3′ end of the SeAP gene, between positions 612 and 737 (125 nucleotides), placed under the control of the CMV promoter. A schematic representation of this plasmid is given in FIG. 3A.

3.2 Plasmid pGJA2 (Plasmid SeAPantisense 5′ End)

The plasmid pGJA2 was constructed by removing the major 3′ portion of the SeAPantisense gene from the plasmid pSeAPantisense using the Sph1 and Nae1 restriction enzymes. The ends were ligated after treatment with the Klenow enzyme which makes the ends blunt. The fragment removed corresponded to the portion between positions 1 and 1936 of the SeAPantisense gene. The remaining portion therefore comprised the first 35 5′-bases of the SeAPantisense gene, between positions 612 and 647 (35 nucleotides), placed under the control of the CMV promoter. A schematic representation of this plasmid is given in FIG. 3B.

3.3 Plasmid pGJA3 (Plasmid SeAPantisense 3′ End)

The plasmid pGJA3 was constructed by removing the major 5′ portion of the SeAPantisense gene from the plasmid pSeAPantisense using the XbaI and PvuII restriction enzymes. The ends were ligated after treatment with the Klenow enzyme which makes the ends blunt. The fragment removed corresponded to the portion between positions 647 and 2139 of the SeAPantisense gene. The remaining portion comprised the last 203 bases in 3′ of the SeAPantisense gene, between positions 1936 and 2139 (203 nucleotides), placed under the control of the CMV promoter. A schematic representation of this plasmid is given in FIG. 3C.

3.4 Plasmid pGJA9 (Plasmid SeAPantisense 5′ and 3′ Ends)

The plasmid pGJA9 was constructed by removing the intermediate portion between the 5′ and 3′ ends of the SeAPantisense gene from the plasmid pSeAPantisense using the DraIII and PvuII restriction enzymes. The ends were ligated after treatment with the Klenow enzyme which makes the ends blunt. The fragment removed corresponded to the portion between positions 737 and 1936 of the SeAPantisense gene. The remaining portion therefore corresponded to the 5′ end and the 3′ end of the SeAPantisense gene, between positions 612 and 737 (the first 125 nucleotides in 5′ of the antisense SeAP gene) and 1936 and 2139 (the last 203 nucleotides in 3′ of the SeAPantisense gene), respectively, these two portions being placed together under the control of the CMV promoter. A schematic representation of this plasmid is given in FIG. 3D.

### Example 4

Construction of Plasmids which Allow the Simultaneous Production of a Transcript and of its Antisense Transcript

4.1 Plasmid pGJA 15-2 (a Single SeAP Coding Sequence Surrounded by a Constitutive Promoter and a Conditional Promoter in the Opposite Direction in 3′)

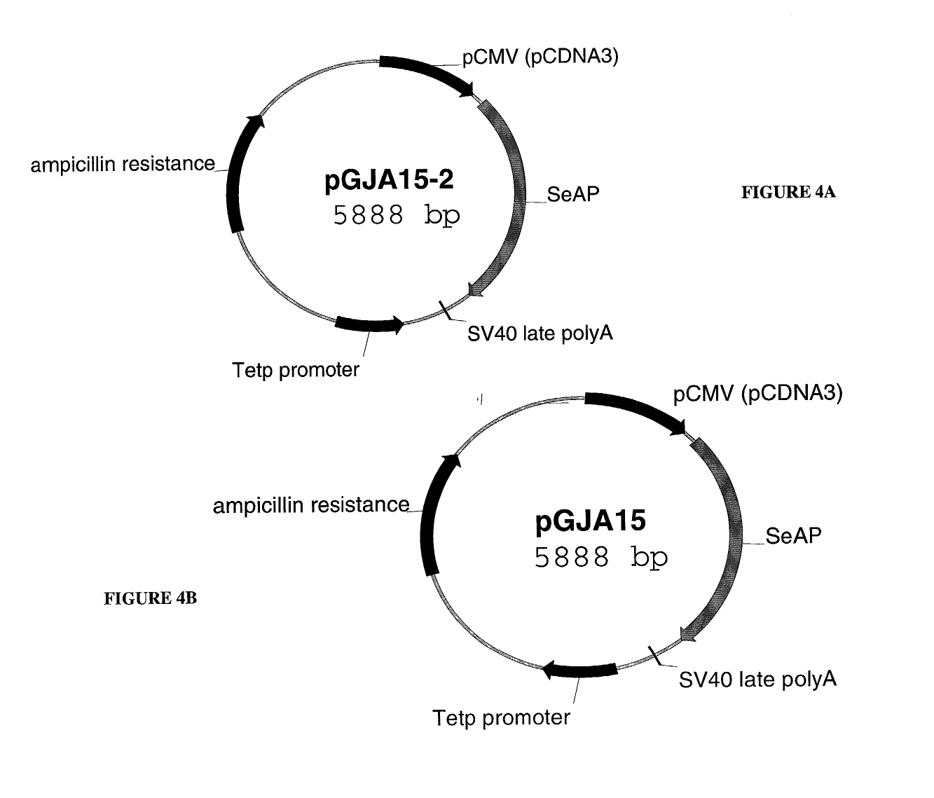

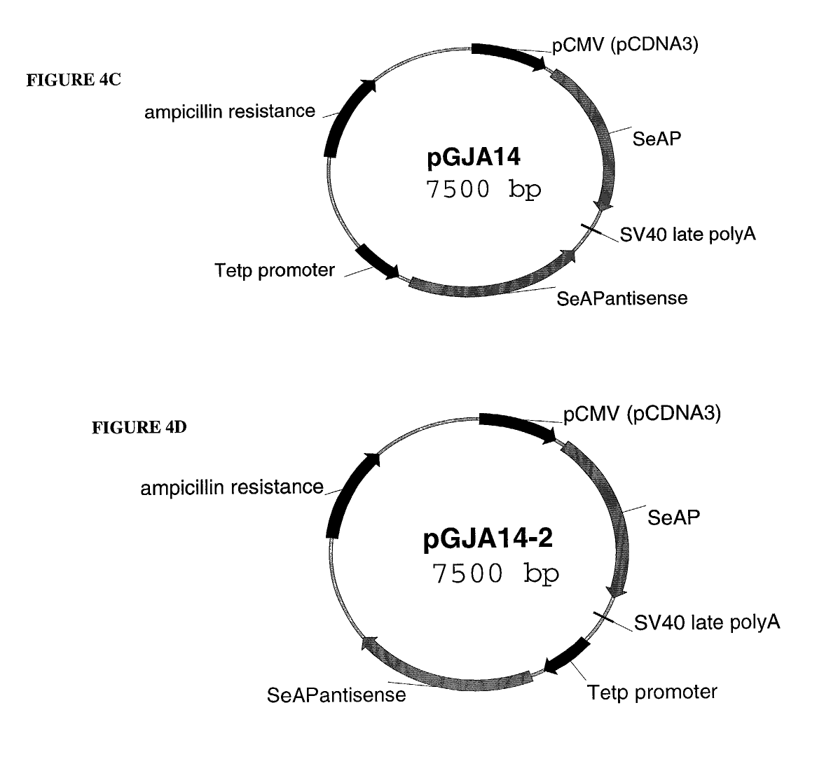

The plasmid was constructed by inserting the tetracycline-repressible promoter (Tetp) into the plasmid pXL 3010 at the Eco47 III restriction site, after the polyA sequence. The Tetp promoter was placed in the opposite direction to that of the CMV promoter which was located upstream of the SeAP gene. In this way, the CMV promoter induces the synthesis of the SeAP transcript constitutively, and the Tetp promoter placed head to tail induces, in the absence of tetracycline, the production of an antisense transcript. In the absence of tetracycline, the SeAP activity was inhibited. A schematic representation of this plasmid is given in FIG. 4A.

4.2 Plamid PGJA15 (a Single SeAP Coding Sequence Surrounded by a Constitutive Promoter and a Conditional Promoter in the Same Direction)

This plasmid was constructed by inserting the same Tetp promoter at the same place as for the plasmid PGJA 15-2, but in the same direction as the CMV promoter which was located upstream of the SeAP gene. This plasmid serves as a control to verify that the Tetp promoter oriented in this way should not modify the expression of SeAP. A schematic representation of this plasmid is given in FIG. 4B.

4.3 Plasmid PGJA14 (constitutive promoter-SeAP and inverted conditional promoter-SeAPantisense, placed in opposite directions)

This plasmid was constructed by inserting a “Tetp promoter+sequence of the SeAPantisense gene” set into the plasmid pXL3010, at the same place as for the plasmid PGJA 15, in the opposite direction to the “CMV promoter+SeAP sequence” set. In this way, the CMV promoter induces the synthesis of the SeAP transcript constitutively, and the Tetp promoter placed in the opposite direction induces, in the absence of tetracycline, the production of the antisense transcript included in the “Tetp promoter+SeAPantisense sequence” set. Under these conditions, the SeAP activity was inhibited, in the absence of tetracycline. A schematic representation of this plasmid is given in FIG. 4C.

4.4 Plasmid PGJA14-2 (Constitutive Promoter-SeAP and Inverted Conditional Promoter-SeAPantisense, Placed in the Same Direction)

This plasmid was constructed by inserting a “Tetp promoter+sequence of the SeAPantisense gene” set into the pXL3010 plasmid, at the same place as for the plasmid PGJA 15, and in the same direction as the “CMV promoter+SeAP sequence” set. In this way, the CMV promoter induces the synthesis of the SeAP transcript constitutively, and the Tetp promoter induces, in the absence of tetracycline, the production of the antisense transcript included in the “Tetp promoter+SeAP antisense sequence”. In the absence of tetracycline, the SeAP activity was inhibited. A schematic representation of this plasmid is given in FIG. 4D.

### Example 5

Construction of hPPARγ2-inducible Plasmids

5.1: Plasmid pSG5-hPPARγ2 (Human Transactivator PPARγ2 Plasmid)

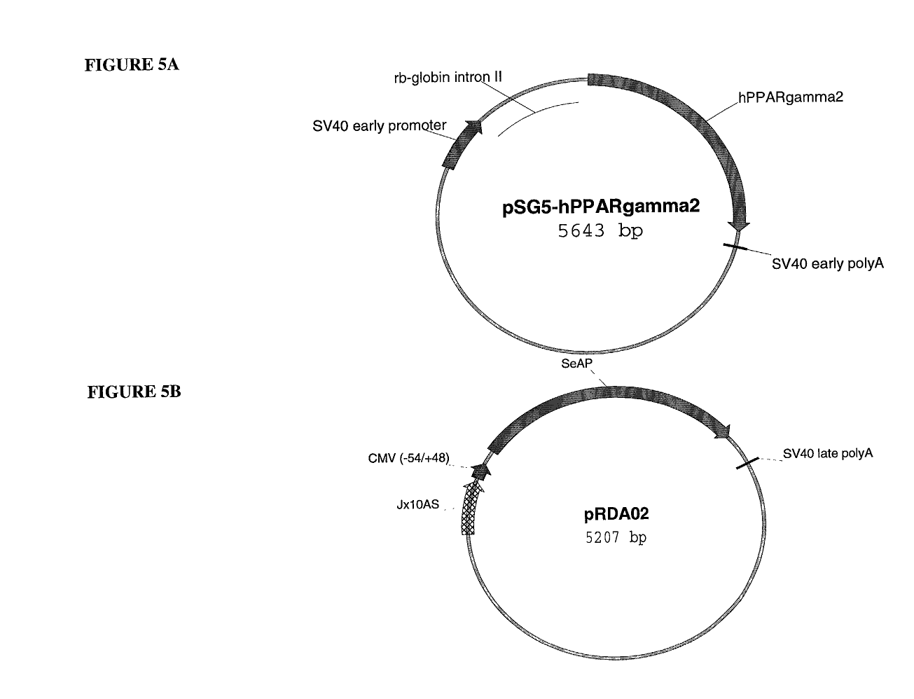

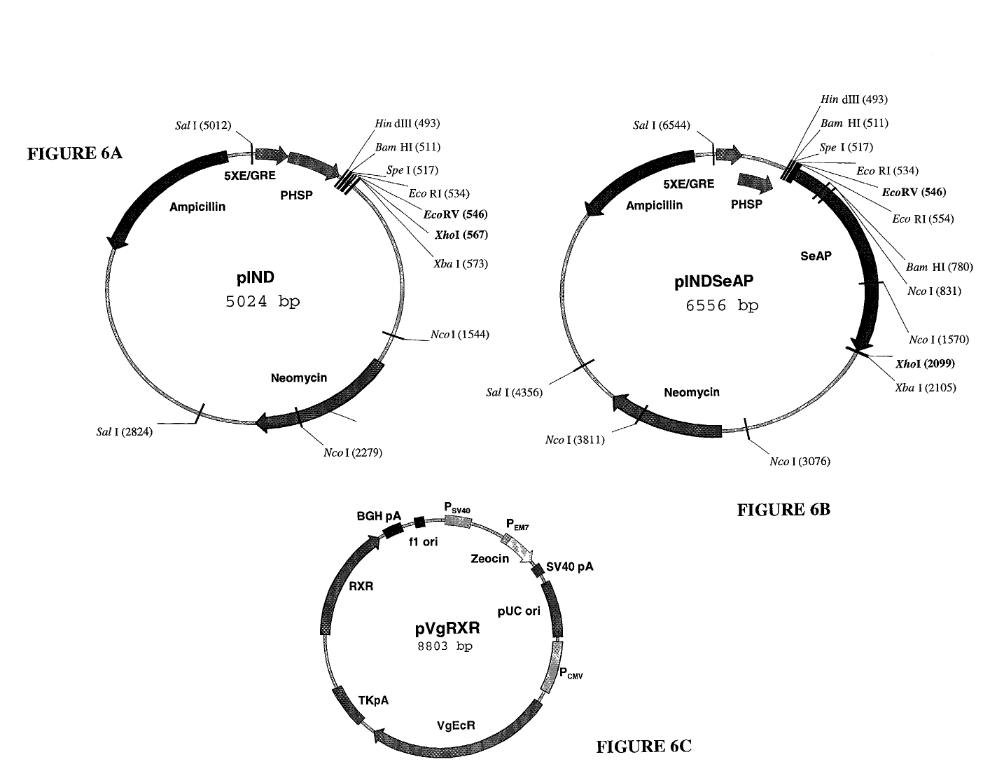

The plasmid pSG5-hPPARγ2 comprised the gene of the transactivator of human origin hPPARγ2, which was capable of activating a minimum promoter comprising, upstream, the J region of the human ApoAII promoter repeated 10 times in reverse orientation (J×10 AS), when it was coexpressed with the plasmid pVgRXR (FIG. 6C) encoding the retinoid receptor RXR. The transactivator was under the control of the SV40 promoter. It was flanked, in its 5′ portion, by an intron from rb-globin (rabbit) and, in its 3′ portion, by a polyA transcription termination sequence from the SV40 virus. A schematic representation of this plasmid is given in FIG. 5A.

5.2: Plasmid pRDA02 (Plasmid SeAP Under the Control of the J×10 AS inducible promoter)

The plasmid pRDA02 comprised the SeAP reporter gene placed under the control of a CMV promoter comprising, upstream, a J×10 AS region which can be induced by the product of the hPPARγ2 gene. The SeAP gene was flanked, in its 3′ portion, by a polyA transcription termination sequence from the SV40 virus. A schematic representation of this plasmid is given in FIG. 5B.

### Example 6

Construction of Ecdysone-inducible Plasmids

6.1: Plasmid pINDSeAP (Promoter Comprising the SeAP Gene Under the Control of the Ecdysone-inducible PHSP Promoter)

The plasmid pINDSeAP was constructed by inserting the gene encoding SeAP between the EcoRI and XhoI restriction sites in the multiple cloning site of the vector pIND (FIG. 6A; InVitrogen). The expression of the gene encoding SeAP was therefore under the control of the ecdysone system uses a heterodimer of the ecdysone receptor (VgECR) and of the retinoid X receptor (RXR). This heterodimer binds to an ecdysone response element (E/GRE on the plasmid IND). The PHSP promoter was a drosophila minimal heat shock promoter (No et al., PNAS 1996, 93:3346-3351). A schematic representation of this plasmid is given in FIG. 6B.

6.2: Plasmid PVgRXR (FIG. 6C; InVitrogen)

The plasmid VgRXR encodes firstly the RXR receptor and, secondly, a VP16/ECR fusion protein. Thus, a heterodimer comprising VP16 can be formed which will activate the transcription in the presence of ecdysone or of analogueues thereof, such as for example Ponasterone A (Pon; FIG. 26) (No et al., PNAS 1996, 93:3346-3351).

### Example 7

Functionality of the Plasmids Comprising a Sequence Encoding an Inhibitory Transcript of Antisense Type in vitro

### Example 7.1

Cell culture

The cells used were NIHT3T3 murine fibroblasts (ATCC: CRL-1658). These cells were seeded 24 h before transfection, in 6- or 24-well plates, at a density of 5×10⁴ cells/well in 1 ml of medium, or of 2.5×10⁵ cells/well in 2 ml. The culture medium used was DMEM™ medium (Life Technologies Inc.) supplemented with 10% of calf serum. The cell cultures were incubated in an incubator at 37° C. in a humid atmosphere and under a partial CO₂ pressure of 5%. The transfections were carried out approximately 24 h after seeding, when 50 to 80% confluence was obtained. C2C12 cells were murine myoblast cells (ATCC: CRL1772) and were cultured on a DMEM™ medium (Life Technologies Inc.) supplemented with 10% of fetal calf serum to which L-glutamine, 2 mM final, and antibiotics, 50 units final of penicillin and 50 μg/ml of streptomycin, were added.

### Example 7.2

Cell Transfection Carried out Using a Cationic Lipofectant Diluted solutions of DNA and of cationic lipid RPR 120535 (Bik G et al., J. Med. Chem, 41 (1998) 224-235) were prepared separately with a view to obtaining for the transfection a concentration of approximately 6 nmol of lipid RPR 120535 B/μg of DNA. Each solution was first diluted in a solution of 20 mM final sodium bicarbonate in 150 mM final NaCl, and incubated for 10 minutes at room temperature (R.T.). The cationic lipid solution was then distributed, volume for volume, into the DNA solutions. A new incubation was carried out for 10 min at R.T., and the complexes formed were then diluted 10-fold in culture medium supplemented with serum. After a final incubation of 10 minutes, the culture medium in the plates was removed and 1 or 2 ml/well of these solutions, depending on whether 24- or 6-well plates were used respectively, were distributed.

### Example 7.3

Measurement of the Luciferase Activity

The luciferase activity was measured 24 h after transfection. Luciferase catalyzes the oxidation of luciferin, in the presence of ATP, of Mg²⁺ and of O₂, with concomitant production of a photon. The total emission of light, measured by a luminometer, was proportional to the luciferase activity of the sample. The culture medium was removed beforehand, the cells were rinsed twice with PBS, and then lyzed for 15 min at room temperature, with 200 μl of Cell Lysis Buffer (Promega Corporation) per well. The Luciferase Assay System™ kit (Promega Corporation) was then used for the activity measurements according to the recommended protocol. The luciferase activity was related to the protein concentration of the cell lysate supernatants. The measurement of the protein concentration of the cell extracts was carried out using the BCA method (Pierce) using bicinchoninic acid (Wiechelman et al., Anal Biochem, 175 (1998) 231-237).

### Example 7.4

Measurement of the SeAP Activity

The SeAP activity was measured on the culture supernatants 48 h after transfection, using the Phospha-Light™ kit (Tropix, Inc.).

### Example 7.5

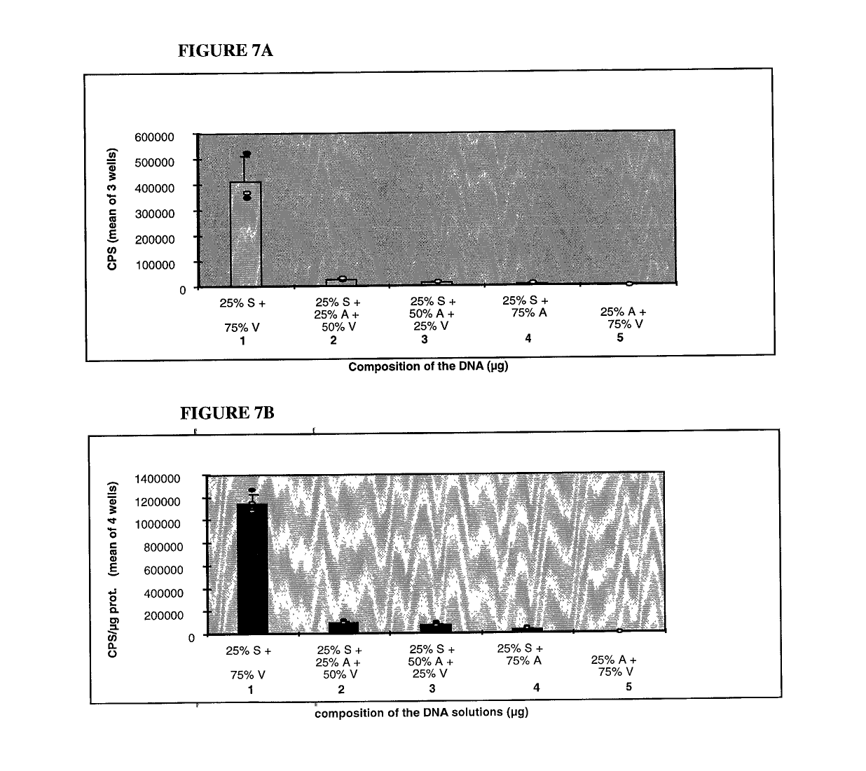

Inhibition in vitro of the Expression of the SeAP (FIG. 7A) or Luciferase (FIG. 7B) Reporter Genes by the Inhibitory Transcript of Antisense Type

The results of the relative activities of luciferase and SeAP under the various conditions of transfection in vitro (FIGS. 7A and 7B) show, firstly, that the luciferase and SeAP reporter genes were well expressed in the NIH 3T3 cells (columns 1 of FIGS. 7A and 7B). Secondly, when the cells were cotransfected with both the sense and antisense plasmids comprising the same reporter gene, the inhibition of the expression was about 90% using a sense/antisense ratio of 1 (columns 2). The degree of inhibition was increased up to 95% and 97% when an antisense/sense ratio of 2 (column 3) or of 3 (column 4) was used. The columns 5 represent the negative control into which sense plasmids encoding SeAP (FIG. 7A) and luciferase (FIG. 7B) were not injected.

### Example 7.6

Verification of the Expression of the Inhibitory Transcript of Antisense Type and of the Sense Transcript in vitro

48 hours after transfection, the total RNAs were prepared by the Trizol method (Gibco BRL) using NIH 3T3 cells. The transcription products from plasmids pXL3010 and pSeAPantisense were revealed by RT-PCR using primers 11 (5′ CGATCATGTTCGACGACGCC3′) (SEQ ID NO: 8) and 12 (5′CCAGGTCGCAGGCGGTGTAG3′) (SEQ ID NO: 9) located at positions 1812-1831 and 2249-2230, respectively, on the plasmid pXL3010, with the aid of the “one step RT-PCR system” kit (Gibco BRL) following the supplier's instructions, and according to the conditions: 40 min at 50° C., then 30 cycles (2 min at 94° C.; 1 min at 94° C.; 1 min at 55° C.; 1 min 30 at 72° C.; termination 3 min at 72° C.).

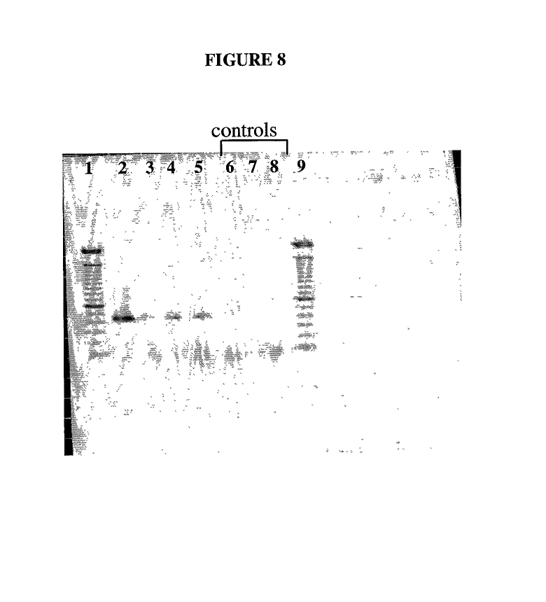

The RT-PCR products were then loaded on to 0.8% agarose gel, and the presence of a band at the expected size of 418 bp was observed (lane 2, FIG. 8) which correctly reflected the transcription of the sense SeAP gene (lane 3, FIG. 8), and of the SeAP sense and SeAP antisense in various proportions, 1:1 (lane 4, FIG. 8) and 1:3 (lane 5, FIG. 8).

Lanes 6 to 8 correspond to negative controls of the experiment in which a PCR without prior reverse transcription was carried out.

### Example 7.7

Specficity of the inhibitory Transcripts of Antisense Type

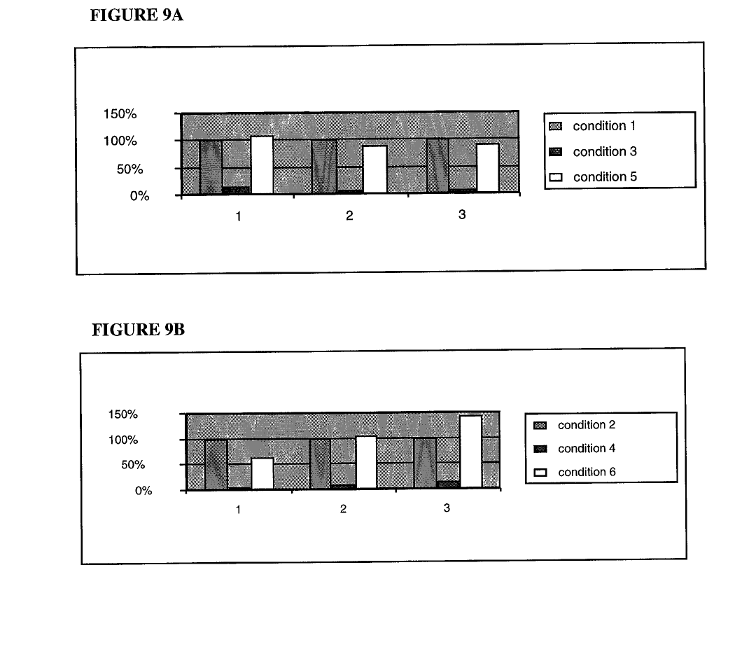

The results of a series of crossed cotransfections of a plasmid encoding SeAP (pXL3010) and of a plasmid encoding the antisense of SeAP (pSeAPantisense) or of luciferase (pLucAntisense), and inversely cotransfections of a plasmid encoding luciferase (pXL3031) and of a plasmid encoding the antisense of SeAP (pSeAPantisense) or of luciferase (pLucAntisense), were given in FIGS. 9A and 9B.

These results clearly demonstrate that there were no aspecific cross reactions, i.e., that the SeAP antisense had no effect on the expression of luciferase, and similarly that the luciferase antisense had no effect on the expression of SeAP. These results also show that the inhibition observed cannot be attributed to the coexpression of any sense and antisense sequences, but, on the contrary, required the coexpression of a transcript which was antisense for a specific sense sequence.

### Example 8

Absence of Inhibition in vivo of the Expression of SeAP by the SeAP Antisense when Injected 22 Days After the Sense SeAP Reporter Gene

### Example 8.1

Electrotransfer into Skeletal Muscle

The 6-week-old SCID mice were first anesthetized with a Ketamine/Xylazine mixture (250 μl/mouse). The various plasmids in solution in 150 mM NaCl were then injected intramuscularly into the tibialis cranialis muscle of the mice. The injection was followed by a series of electrical pulses: 8 pulses of 20 ms, 200 V/cm, 1 Hz (Mir et al., PNAS, 96 (1999) 4262-67). The amount of circulating SeAP was regularly monitored by taking blood samples and assaying the phosphatase activity using the Phospha-Light kit (Tropix).

### Example 8.2

Comparison of the Percentage of Inhibition for the Inhibitory Transcript of Antisense Type when it was Coinjected with the SeAP Reporter Gene or Postinjected 22 Days After the Injection of the SeAP Gene

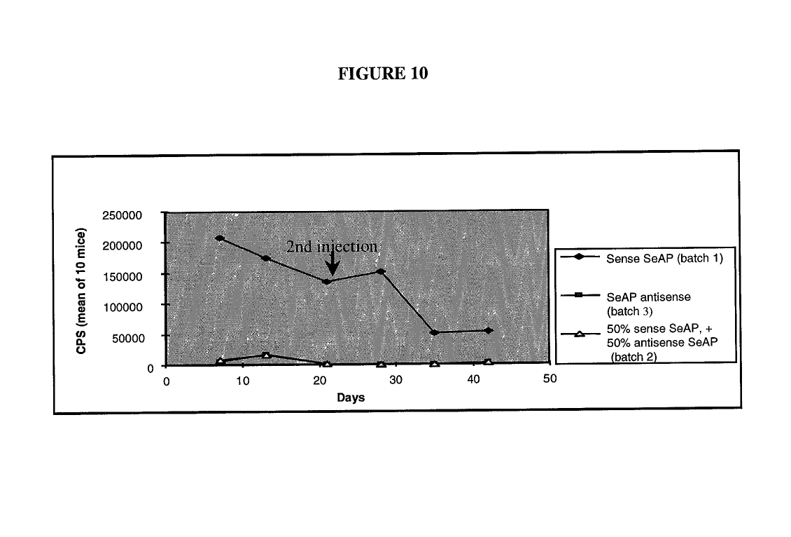

The results, given in FIG. 10, show that the injection of the pSeAPantisense plasmid did not lead to effective inhibition of the SeAP reporter gene (pXL3010) injected 22 days beforehand. For example, more than 20 days after the injection of the antisense transcript, the expression of SeAP observed had decreased by only 60% (batch 1, FIG. 10).

This clearly indicates, therefore, that the antisense transcript could not effectively inhibit the previously administered exogenous SeAP gene, although it has been recognized that the latter remains stable and functional for approximately 9 months after injection and electrotransfer in vivo (Mir et al., PNAS, 96(8) (1999) 4262-4267; Mir et al., C R Acad Sci III, 321(11) (1998) 893-899). Approximately 30% residual expression of the exogenous SeAP gene was in fact observed.

On the other hand, FIG. 10 clearly shows that a coinjection of the inhibitory transcript of antisense type and of the sense sequence of the exogenous SeAP reporter gene conferred very strong inhibition of the expression of SeAP, since no residual expression of this gene could be detected. The coexpression of the sense and antisense SeAP gene makes it possible to abolish the expression of the SeAP reporter gene in vivo (batch 2, FIG. 10).

The injection of antisense alone, as a control, conferred no activity (batch 3, FIG. 10).

### Example 8.3

Verification of the Expression in vivo of the Sense and Antisense Transcripts

The muscles of the mice were removed and ground, and the total RNAs were extracted. RT-PCR reactions were carried out following the protocol described above in Example 3.6. The reaction products were separated on agarose gel and visualized with ethidium bromide.

A photograph of this gel, which is given in FIG. 11A, shows that both the sense and antisense RNA were expressed in the muscles of mice which have undergone a first injection of plasmids pXL3010, and a subsequent injection of plasmid carrying the sequence of the inhibitory transcript of antisense type, pSeAPantisense (lanes 2 and 3).

Conversely, when a coinjection of pXL3010 and pSeAPantisense was carried out, only the antisense RNA was present; the SeAP mRNA was not detected (lanes 4 and 5). This confirms the effectiveness of the inhibition obtained by coinjection of the sense sequence and of its antisense inhibitory transcript.

When the plasmid pSeAPantisense was injected alone, as a control, the SeAP antisense RNA only was detected (lanes 6 and 7).

The agarose gel was transferred on to a Hybond N+ nylon membrane (Amersham) and hybridized with a ³²P-labelled oligonucleotide probe specific for the sense and antisense transcripts of the SeAP reporter gene. The membrane was then exposed on an X-ray film, and the film was developed three hours later. A photograph of this film, which was given in FIG. 11B, confirmed the above results. Specifically, the presence of a product of transcription of the SeAP reporter gene was not detected in lane 4, which corresponded to the coinjection of the plasmids comprising the sense sequence of the reporter gene (pXL3010) and the antisense sequence (pSeAPantisense), whereas the product of transcription of the SeAP reporter gene was detected in lane 2, which corresponded to the experiment of postinjection of these same plasmids.

### Example 8.4

Monitoring of the Circulating SeAP Relative Activity in vivo After Injection of the Plasmid Comprising the Sense Sequence of the SeAP Gene (pXL3010), Followed by a Postinjection of the Plasmid Comprising the Sequence of the Inhibitory Transcript of Antisense Type of the SeAP Reporter Gene (pSeAPantisense)

50 six-week-old female SCID mice, divided into 5 groups of 10, and were treated as described above in Examples 3.2 and 3.3.

The results given in FIG. 12 show clearly, and in a reproducible manner, that no inhibition effect can be demonstrated when the procedure was carried out by injecting firstly the sequence encoding the sense transcript and then, secondly, encoding the inhibitory transcript of antisense RNA type.

### Example 8.5

Monitoring of the Circulating SeAP Relative Activity in vivo After Coinjection of the Plasmids pXL3010 and pSeAPantisense

50 six-week-old female SCID mice, divided into 5 batches of 10, and were treated as described above in Examples 3.2 and 3.3.

The results, given in FIG. 13, show that the coinjection of these two plasmids (batch 3) made it possible to obtain very low, or even zero, expression of the exogenous SeAP reporter gene, indicating that the inhibitory transcript of antisense RNA type acted by strongly inhibiting the transcription of the SeAP reporter gene with which it was coinjected, this being in a constitutive way over a variable period of time ranging from 7 to 85 days after the coinjection. Control batches 1, 2, 4 and 5, which correspond to an injection of the plasmid carrying the sense sequence of the reporter gene alone, showed expression of the gene at varying levels throughout the evaluation period.

### Example 9

Functionality of the Inhibition of the Inhibitory Transcript of Antisense Type when it was Placed Under the Control of a Tetracycline-repressible Promoter, and Measurement of Inhibition in vitro

### Example 9.1

Functionality in vitro of the Tetracycline-repressible Promoter

The experiments were carried out on NIH 3T3 cells, with the SeAP and luciferase reporter genes, these two reporter genes having been described above.

### Example 9.2

Regulation of the SeAP Reporter Gene in vitro with an Inhibitory Transcript of the Antisense Type

The results, given in FIG. 14A, show that the inhibitory transcript of antisense type under the control of a CMV strong constitutive promoter (pSeAPantisense), coexpressed with the sense sequence of the SeAP reporter gene in a proportion of 0.5 and 1, conferred respectively 70% to 83% inhibition of the expression of the gene in vitro (columns 2 and 3). On the other hand, when the inhibitory transcript of antisense type was placed under the control of the tetracycline promoter (pTetSeAPantisense), the inhibition in vitro was weaker and incomplete, from 45% to 60%, respectively, in the same ratios (columns 4 and 6).

In the presence of an external repressor agent such as tetracycline, induction of the expression of SeAP was observed (columns 5 and 7).

The results, given in FIG. 14B, show partial inhibition of the SeAP reporter gene when it was coinjected with the plasmid comprising the sequence of the inhibitory transcript of antisense type of the SeAP gene under the control of the CMV promoter (pSeAPantisense), in a 1:1 proportion (column 2), or under the control of the tetracycline-repressible promoter (columns 3 and 6), with respect to the level of expression of the SeAP reporter gene measured after injection of the plasmid comprising the sense sequence of SeAP (pXL3010) (columns 1 and 5).

The administration of tetracycline made it possible to reestablish very satisfactory expression of the SeAP reporter gene (columns 4 and 7).

### Example 9.3

Regulation of the Luciferase Reporter Gene in vitro with an Inhibitory Transcript of Antisense Type

The results given in FIG. 15 demonstrate, first of all, the functionality in vitro of the plasmids comprising the sense and antisense sequence of the luciferase reporter gene under the control of the tetracycline-repressible promoter (pTetLucAntisense, pTetLuc and pTetSpliceAntisense).

In the absence of tetracycline, the inhibitory transcript of antisense type was expressed and resulted in incomplete inhibition of 60-70% (columns 3 and 6), whereas, when the inhibitory transcript of antisense type was placed under the control of the CMV promoter, the inhibition was about 90%, using a sense/antisense ratio of 1:1 (column 2).

In the presence of tetracycline, the expression of the luciferase reporter gene was restored to a satisfactory level (columns 4 and 7), with respect to the level of luciferase obtained by transfection of a single plasmid comprising the sense sequence of the luciferase reporter gene (pXL3031) (column 1). These results show that it was possible to regulate indirectly the expression of exogenous reporter genes in the presence of an external agent which was a repressor of the inhibitory transcript, such as tetracycline.

### Example 10

Measurement of Strong Inhibition in vivo with an Inhibitory Transcript of Antisense Type Placed Under the Control of a Repressible Promoter

40 SCID mice were treated as described above, using the plasmids pXL3010, pSeAPantisense, pTetSeAPantisense and pTet-tTAk.

The results, given in FIG. 16A, clearly show, unlike the results of inhibition in vitro, and with respect to the level of expression in vivo of the SeAP reporter gene (batch 1), that effective inhibition of the expression of SeAP was obtained when the plasmid comprising the sense sequence of the SeAP reporter gene (pXL3010) and the plasmid comprising the SeAP antisense sequence under the control of a CMV strong promoter (pSeAPantisense) (batch 2) were coinjected and coexpressed, alternatively when coinjecting and coexpressing the plasmid comprising the sense sequence of the SeAP reporter gene (pXL3010) and the plasmid comprising the antisense sequence of SeAP under the control of the tetracycline-repressible promoter (pTetSeAPantisense) (batch 3).

### Example 11

Regulation in vivo with an Inhibitory Transcript of Antisense Type Placed Under the Control of a Repressible Promoter

The results given in FIG. 16B show that the coinjection of the plasmids carrying the sense sequence of the SeAP reporter gene (pXL3010) and the antisense sequence of the gene under the control of the tetracycline-repressible promoter (pTetSeAPantisense), in the presence of an external repressor agent, such as tetracycline, made it possible to obtain a satisfactory biological level of the SeAP reporter gene (batch 3, D8).

Inhibition of the expression of the exogenous SeAP reporter gene could again be observed when the administration of tetracycline was stopped on the 10th day (batch 3: D15, D22, D30 and D63). These results also confirm that this inhibition was reversible, since the administration of a repressor agent which was a tetracycline analogue, doxycycline, on the 63rd day made it possible to reestablish expression of the SeAP reporter gene (batch 3: D70).

### Example 12

Regulation with an Inhibitory Transcript of Ribozyme Type

As described above for the construction of the plasmids pTetSeAPantisense and pTetLucAntisense, a plasmid which comprises a hammerhead ribozyme sequence is constructed by cloning a sequence comprising at least one GTC site, chosen at positions 958, 1058, 1127, 1205, 1243, 1600, 1620, 1758, 1773, 1880, 1901, 1988, 2007, 2085 and 2201 on the plasmid pXL3010 (SeAP reporter gene), downstream of the tetracycline-repressible promoter TetRS, into the previously digested plasmid pTet-Splice (Gibco BRL), so as to give the plasmid pTetSeAPribozyme.

30 six-week-old SCID mice are treated as described above in Example 4, and are divided into three groups of 10.

The first group is treated as described above with the plasmid pXL3010.

The second group receives the plasmids pXL3010, pTet-tTAk and pTetSeAPribozyme by coinjection. The third group is treated like group 2, and the mice are given a drink comprising doxycycline (400 mg/l). The circulating SeAP level is monitored as described above.

In the second group, after coinjection and electrotransfer of the plasmid comprising the sense sequence of the SeAP reporter gene (pXL3010) and of the plasmid comprising the sequence of the ribozyme inhibitory transcript specific for SeAP under the control of a tetracycline-repressible promoter (pTetSeAPribozyme), effective inhibition of the expression of SeAP is observed, with respect to the observed expression of the SeAP reporter gene in the first group of mice tested, indicating that the inhibitory transcript of ribozyme type is capable of strongly inhibiting in vivo the transcription of the exogenous SeAP gene with which it is coadministered.

The oral administration of a tetracycline analogue, doxycycline, as a repressor agent, makes it possible to restore the expression of SeAP.

### Example 13

Regulation with an Inhibitory Transcript of Antisense Type Comprising an Aptamer Sequence

The plasmid pSeAPantisense (FIG. 1C) as described in Example 1.3 is modified in order to insert, at the 5′ end of the sequence of the antisense inhibitory transcript, a ligand-dependent aptamer sequence, having the sequence 5′ GGCCUGGGCGAGAAGUUUAGGCC 3′ (SEQ ID NO: 10), recognized by neomycin B as described by Cowan et al. (Nucleic Acids Res., 28 (15) (2000) 2935-2942), so as to give the plasmid designated pSeAPaptamerAS.

30 six-week-old SCID mice are treated as described above in Example 4, and are divided into three groups of 10.

The first group is treated as described above with the plasmid pXL3010. The second group receives the plasmids pXL3010 and pSeAPaptamerAS by coinjection followed by electrotransfer. The third group is treated like group 2, and also receives an IP injection of neomycin B in a proportion of approximately 500 μg/mouse. The circulating SeAP level is then monitored as described above. While for the first group, constant expression of the SeAP reporter gene is detected, in the second group, effective inhibition of the SeAP gene by the inhibitory transcript comprising an aptamer sequence is observed.

Expression of SeAP can be restored in the third group, to which an effective amount of neomycin B which recognizes the aptamer sequence carried by the plasmid pSeAPaptamerAS is administered. A large decrease in the circulating SeAP level, and therefore inhibition of the expression of the SeAP reporter gene, can again be observed when the administration of neomycin B is stopped.

### Example 14

Regulation with an Inhibitory Transcript of Ribozyme Type Comprising an Aptamer Sequence

A plasmid which comprises a hammerhead ribozyme sequence is constructed by cloning a sequence comprising at least one GTC site, chosen at positions 958, 1058, 1127, 1205, 1243, 1600, 1620, 1758, 1773, 1880, 1901, 1988, 2007, 2085 and 2201 on the plasmid pXL3010, downstream of the CMV promoter, into the previously digested plasmid pXL3296 (Soubrier et al.), so as to give pSeAPribozyme. The latter is then modified in order to insert, at the 5′ end of the sequence of the inhibitory transcript of ribozyme type, an aptamer of sequence 5′GGUGAUCAGAUUCUGAUCCAAUGUUAUGCUUCUCUGCCUGGGAACAGCUG CCUGAAGCUUUGGAUCCGUCGC 3′ (SEQ ID NO: 11), as described by Werstuck et al. Science, 282 (1998), 296-298, and recognized by the Hoechst 33258 dye (H33258), so as to give the plasmid designated pSeAPaptazyme.

Three groups of 10 six-week-old SCID mice are treated: the first group receives the plasmid pXL3010 by injection followed by electrotransfer, the second group receives the plasmids pXL3010 and pSeAPaptazyme, also by coinjection followed by electrotransfer, and finally, the third group is treated like group 2, but also receives, via the drinking water, an amount of H33258 dye (400 mg/l). The monitoring of the circulating SeAP level shows effective inhibition in vivo of SeAP activity, which is restored to a significant level in the third group of mice, which receive the H33258 dye or ligand specific for the aptamer sequence present in the plasmid pSeAPaptazyme.

### Example 15

In vitro Inhibition of the Expression of the SeAP Reporter Genes with Shorter Fragments of the Inhibitor Transcript SeAPantisense

### Example 15.1

Inhibition Obtained with the Plasmids pGJA1, pGJA2 and pGJA3

(transcript fragment comprising, respectively, the first 125 and the first 35 bases in 5′ of the sequence of the SeAPantisense gene, and the last 203 bases in 3′ of the sequence of the SeAPantisense gene)

Measuring the SeAP activity under the various conditions for in vitro transfection made it possible to compare the inhibitory effect of the subfragments of the SeAPantisense transcript with those of the whole SeAPantisense transcript. The results of FIG. 17 show that, without reaching the inhibition observed for the whole antisense transcript pSeAPantisense (column 2), the 125- or 35-nucleotide fragments of the 5′ end of the SeAPantisense transcript, carried by the plasmids pGJA1 and pGJA2, and also the 203-nucleotide fragment of the 3′ end of the SeAPantisense transcript, carried by the plasmid pGJA3, produced significant inhibition of the SeAP activity measured in NIH3T3 cells (columns 3, 4 and 5 of FIG. 17, respectively).

### Example 15.2

Inhibition Obtained with the Plasmid pGJA9 (Transcript Fragments Comprising Both the 5′ End and 3′ End of the SePantisense Transcript)

The inhibition caused by the fusion of both the 3′ end (203 nucleotides) and 5′ end (125 nucleotides) of the SeAPantisense transcript is represented in columns 7 and 8 of FIG. 18. This transcript was produced from the plasmid pGJA9. It significantly inhibited the SeAP activity measured in the cells, by comparison with the maximum inhibition attained with the whole SeAPantisense transcript (columns 5 and 6). The results obtained with the shorter fragments, either from the start (pGJA1 and pGJA2), from the end (pGJA3) or from the fusion of the sequence of the start and of the end of the SeAPantisense gene (pGJA9), clearly showed significant levels of inhibition of the activity of the SeAP transgene could be obtained using shorter portions of the inhibitory transcript. A summarizing table of the percentages of inhibition obtained using the four plasmids pGJA1, pGJA2, pGJA3 and pGJA9 is given in FIG. 19.

### Example 16

Kinetics of Regulation, in vivo, with the Inhibitory Transcript of SeAPantisense Type Placed Under the Control of a Doxycycline-repressible Promoter.

The results given in FIG. 20 establish the effectiveness of a regulation system similar to that described in Example 7, in which tetracycline has been replaced with an analogue, doxycycline. The SCID mice were treated as previously described. The induction of the expression of the exogenous SeAP reporter gene was obtained over two timescales. In the case of batch 3, the mice drank water except on day 170, at which time they drink doxycycline. There was zero expression of SeAP in the absence of doxycycline, and a very slight increase in this expression was observed after doxycycline had been taken for one day.

In batch 4, the mice drank doxycycline for 7 days, followed by breaks of 20, 30 or 40 days. Taking doxycycline for a week this time caused considerable increases in the expression of SeAP, which regressed significantly during the periods when water was taken.

### Example 17

Verification of the Functionality of the Plasmids pGJA14, pGJA14-2, pGJA15 and pGJA15-2 for Expressing SeAP

The expression of SeAP by the transcripts encoded by each of the plasmids pGJA14, pGJA14-2, pGJA15 and pGJA15-2 was evaluated using a series of experiments carried out in the absence of the transactivator tTA. FIG. 21 shows the levels of expression of SeAP compared with those produced by the plasmid pXL3010. There was notable expression, greater than that of the SeAP comprised by the plasmid pXL3010. The plasmids indeed allowed the expression of SeAP.

### Example 18

Regulation of the Expression of SeAP by the Plasmids pGJ14, pGJ15 and pGJA15-2 Coinjected with the Plasmid pTet-tTAk

### Example 18.1

Regulation of the Expression of SeAP by the Plasmids pGJA15 and pGJA15-2 Coinjected with the Plasmid pTet-tTAk

The results presented in FIG. 22 evaluate the inhibition of the expression of SeAP on cells cotransfected with the plasmid pTet-tTAk and, respectively, the plasmids pGJA15 and pGJA15-2. In the case of the plasmid pGJA15, in which the orientation of the pTet promoter does not allow the synthesis of the SeAPantisense transcript, no inhibition was observed either in the presence or absence of tetracycline. On the other hand, the plasmid pGJA15-2, in which the pTet inducible promoter was functionally linked to the SeAPantisese gene, produced significant inhibition of the expression of SeAP in the absence of tetracycline. In the presence of tetracycline, partial restoration of SeAP was observed. These results showed that the plasmid pGJA1 5-2 may be used for a strategy for regulating the expression of an exogenous reporter gene, which was based on the coinjection of two plasmids and in which the antisense and the sense were carried on the same plasmid and were produced from the same sequence on the same plasmid.

### Example 18.2

Regulation of the Expression of SeAP by the Plasmid pGJA14 Coinjected with the Plasmid pTet-tTAk

The same experiment as that described in Example 16.1 was conducted on cells cotransfected with the plasmids pGJA14 and pTet-tTAk (FIG. 23). Columns 6 and 7 show that the expression of SeAP was inhibited in the absence of tetracycline, by comparison with the constitutive expression of column 5. This inhibition was partially lifted by adding tetracycline which prevented the transactivator tTA from activating the pTet promoter. These experiments therefore reveal another regulation system based on the coinjection of two plasmids, in which the antisense and the sense were carried on the same plasmid, but produced from two distinct sequences.

### Example 19

Reduction of the Residual Expression of the SeAP Gene in the Context of the hPPARγ2 Inducible System, by Adding Antisense Transcripts of the SeAPantisense Gene

The data presented in FIG. 24 show that the expression of the exogenous SeAP reporter gene (plasmid pRDA02) in the presence of the transactivator hPPARγ2 (plasmid pSG5-hPPARγ2), but in the absence of the BRL fibrate (RPR131300A at 10⁻²M in water) was not zero (column 1). The data of the subsequent columns (columns 3, 5, 7) show that this basic level could be reduced by adding increasing amounts of antisense transcript obtained by transfecting the plasmid pSeAPAS. Moreover, the presence of the antisense transcript did not prevent a certain inducibility of the expression of SeAP by the fibrate (ratios of columns 3 and 4; 5 and 6; 7 and 8; respectively). The combined system of the three plasmids pRDA02, pSG5-hPPARγ2 and pSeAPAS therefore allowed the expression of the exogenous SeAP reporter gene to be controlled while at the same time minimizing expression from residual leaking in the absence of the inducer agent, such as fibrate.

### Example 20

Reduction of the Residual Expression of the SeAP Gene in the Context of an Ecdysone-inducible System, by Adding Antisense Transcripts

FIG. 25 shows, in columns 1 and 2, the level of expression of the SeAP gene carried by the plasmid pINDSeAP, in the presence and absence of an inducer of the ecdysone system, ponasterone (FIG. 26; No et al., PNAS, 1996, 93:3346-3351). In the absence of ecdysone inducer, the level of expression was low, but not zero. This level was taken to zero when the plasmid pSeAPAS, expressing the antisense transcript of SeAP, was cotransfected with the plasmid pINDSeAP (column 3). The combined system of the three plasmids pINDSeAP, pVgRXR and pSeAPAS therefore allowed the expression of the exogenous SeAP reporter gene to be controlled while at the same time eliminating expression from residual leaking observed in the absence of ecdysone inducer.

### Example 21

Kinetics of Regulation, in vivo, with the Inhibitory Transcript of SeAPantisense Type Placed Under the Control of a Doxycycline-repressible Promoter

Thirty 6-week-old SCID mice were treated as previously described and divided up into 5 batches.

The first batch of mice (batch 1; FIG. 27) was treated with the plasmid pXL3010. Residual expression of the SeAP gene was noted when the latter was placed under the control of the constitutive CMV promoter.

The second batch of mice (batch 2; FIG. 27) received, by coinjection followed by electrotransfer, the plasmids pGJA14 and pTet-tTAk. The results given in FIG. 27 clearly show that zero residual expression of the SeAP gene in vivo, in the absence of doxycycline. This establishes the effectiveness of the inhibition of the SeAP gene resulting from the use of a plasmid such as pGJA14, which comprised the SeAP gene under the control of the constitutive CMV promoter and the sequence encoding SeAPantisense under the control of a conditional Tetp promoter in the opposite direction on the same vector.

The third batch of mice (batch 3; FIG. 27) received, by coinjection followed by electrotransfer, the plasmids pGJA14 and pTet-tTAk and doxycycline in the drinking water. The expression of the SeAP gene, measured on the 8th day, was then significantly activated in the presence of doxycycline, at a level which was clearly greater than the constitutive level of expression of SeAP obtained for batch 1.

The fourth batch of mice (batch 4; FIG. 27) received, by coinjection followed by electrotransfer, the plasmids pGJA15-2 and pTet-tTAk. In the absence of doxycycline, the residual expression of SeAP was greatly reduced compared to the constitutive expression observed in batch 1, but not zero. Specifically, residual expression of SeAP was observed when coexpressing, on complementary strands of the same vector, the SeAP gene and the sequence of the antisense transcript, compared to the use of a plasmid comprising both sequences on the same strand of the same vector (batch 2).

The fifth batch of mice (batch 5; FIG. 27) received, by coinjection followed by electrotransfer, the plasmids pGJA15-2 and pTet-tTAk and doxycycline in the drinking water. As for batch 3, in the presence of doxycycline, the expression of SeAP measured on the 8th day was significantly activated.

These results clearly show that inhibition of the expression of the SeAP gene could be obtained when the latter was administered on the same vector as the sequence of the antisense inhibitory transcript, whether on the same strand or on complementary strands. This inhibition is, moreover, clearly reversible when an external agent which inhibits the antisense transcript was administered.

What is claimed is:

1. A method for regulating the expression of a transgene of interest in vivo comprising:
simultaneously introducing into a target nonhuman animal tissue or cell a first nucleic acid comprising the sequence of a transgene of interest encoding a transcript of interest, and a second nucleic acid comprising the sequence of an inhibitory transgene encoding an inhibitory transcript specific for the transcript of interest, wherein each of the sequences are under the control of a transcriptional promoter, and the activity of the inhibitory transcript is optionally regulated with at least one external agent, and the activity of the transcript of interest is optionally regulated with at least one external agent, and
coexpressing said nucleic acids in the target tissue or cell to constitutively inhibit the activity of the transcript of interest with the inhibitory transcript.

2. The method according to claim 1, further comprising administering to the target nonhuman animal tissue or cell at least one external agent which causes the activity of said inhibitory transcript to be inhibited, for the purpose of restoring the activity of the transcript of interest.

3. The method according to claim 2, further comprising administering to the target nonhuman animal tissue or cells at least one external agent which causes the activity of said transcript of interest to be increased, for the purpose of restoring the activity of the transcript of interest.

4. The method according to claim 1, further comprising administering to the target nonhuman animal tissue or cells at least one external agent which causes the activity of said transcript of interest to be increased, for the purpose of restoring the activity of the transcript of interest.

5. The method according to claim 1, wherein the inhibitory transcript is under the control of a repressible promoter, and at least one external agent causes the inhibition of said promoter.

6. The method according to claim 5, wherein the promoter comprises at least one repeat of the tetracycline response operator, and at least one external agent is an external repressor agent chosen from tetracycline and tetracycline analogues.

7. The method according to claim 1, wherein the nucleic acid encoding the inhibitory transcript also comprises an activatable autocatalytic aptamer sequence, and the method comprises at least one external agent which is a specific ligand of the aptamer and which is an activator of the aptamer.

8. The method according to claim 1, wherein the nucleic acid encoding the inhibitory transcript also comprises a sequence which can be recognized by a ribozyme with ligand-dependent activity, and the external agent is a ligand which is an activator of the catalytic activity of the ribozyme.

9. The method according to claim 1, wherein the sequence encoding the transcript of interest is placed under the control of an inducible promoter, and the method comprises at least one external agent which causes the activation of said promoter.

10. The method according to claim 9, wherein the inducible promoter comprises a PPARα response element, and wherein at least one external agent is a PPARα ligand.

11. The method according to claim 10, wherein the inducible promoter comprises a PPARα response element, and at least one external agent is a PPARα ligand chosen from fibrates and fibrate analogues.

12. The method according to claim 9, wherein the inducible promoter comprises a PPARγ response element, and the method comprises at least one external agent which is a PPARγ ligand.

13. The method according to claim 12, wherein the inducible promoter comprises a PPARγ response element, and at least one external agent is a PPARγ ligand chosen from fatty acids and thiazolidinediones.

14. The method according to claim 1, wherein the inhibitory transcript is in the form of an antisense RNA which is complementary to at least one coding portion of the mRNA of the transgene of interest, and the inhibitory transcript is capable of forming with the at least one coding portion of the mRNA of the transgene of interest a Watson and Crick-type linkage.

15. The method according to claim 1, wherein the inhibitory transcript is in the form of an antisense RNA which is complementary to at least one noncoding portion of the mRNA of the transgene of interest, and the inhibitory transcript is capable of forming with the at least one noncoding portion of the mRNA of the transgene of interest a Watson and Crick-type linkage.

16. The method according to claim 15, wherein the inhibitory transcript is in the form of an antisense RNA which is complementary to at least one noncoding portion at the 5′ end of the mRNA of the transgene of interest, and the inhibitory transcript is capable of forming with the at least one noncoding portion at the 5′ end of the mRNA of the transgene of interest a Watson and Crick-type linkage.

17. The method according to claim 15, wherein the antisense RNA is at least 10 ribonucleotides long.

18. The method according to claim 1, wherein the inhibitory transcript is in the form of an RNA capable of forming a triple helix with a portion of the nucleic acid comprising the sequence of the transgene of interest.

19. The method according to claim 1, wherein the inhibitory transcript is in the form of a ribozyme.

20. The method according to claim 1, wherein the transgene of interest encodes a protein of therapeutic interest.

21. The method according to claim 1, wherein the transgene of interest corrects a genetic abnormality or of deficiency.

22. The method according to claim 1, wherein the transgene of interest encodes an activator which is involved in the expression of another gene.

23. The method according to claim 1, wherein the transgene of interest produces a transcript of interest having therapeutic activity.

24. The method according to claim 1, wherein the nucleic acids are carried by a single vector or different vectors.

25. The method according to claim 24, wherein the nucleic acids are carried by the same strand of the same vector.

26. The method according to claim 24, wherein the vector is a plasmid, a cosmid, an artificial chromosome or a nonencapsidated DNA.

27. The method according to claim 24, wherein the vector is a recombinant virus.

28. The method according to claim 27, wherein the recombinant virus is an adenovirus, a retrovirus, a herpesvirus, an adeno-associated virus, a phage or a derivative of these.

29. The method according to claim 24, wherein the vector is a bacterium or parasite.

30. The method according to claim 1, wherein the nucleic acids are introduced into the target tissue or cell using a physical or mechanical method.

31. The method according to claim 30, wherein the nucleic acids are introduced into the target tissue or cell using a physical or mechanical method chosen from injection, ballistic technique, electroporation, sonoporation, exposure to an electrical field, exposure to microwaves, exposure to heat or pressure, or by a combination of these techniques.

32. The method according to claim 30, wherein the nucleic acids are introduced into the target tissue or cell by injection, electrotransfer, or a combination thereof.

33. The method according to claim 1, wherein the nucleic acids are introduced into the target tissue or cell in a form which is complexed with at least one chemical agent or biochemical agent.

34. The method according to claim 33, wherein the at least one chemical agent or biochemical agent is a cationic protein chosen from histones and protamines.

35. The method according to claim 33, wherein the at least one chemical agent or biochemical agent is a polymer chosen from DEAE-dextran, polyamidoamines, polylysines, polyethyleneimines, polyvinylpyrrolidones or polyvinyl alcohols.

36. The method according to claim 33, wherein the nucleic acids are incorporated into lipids in crude form or in the form of liposomes.

37. The method according to claim 33, wherein the nucleic acids are incorporated into nanoparticles.

38. The method according to claim 1, wherein the target tissue or cell is of nonhuman animal or human origin.

39. The method according to claim 38, wherein said target tissue or cell of nonhuman animal or human origin is a muscle cell or a skeletal muscle tissue.

40. The method according to claim 1, wherein the target tissue or cell is of plant origin.

41. The method according to claim 30, wherein the nucleic acids are injected systemically.

42. The method according to claim 41, wherein the nucleic acids are injected intra-arterially or injected intravenously.

43. The method according to claim 42, wherein the nucleic acids are present in a composition comprising pharmaceutically acceptable excipients for the intra-arterial or intravenous methods of administration.

44. The method according to claim 30, wherein the nucleic acids are administered parenterally, topically, cutaneously, vaginally, intranasally, subcutaneously, intra-ocularly, or a combination thereof.

45. The method according to claim 44, wherein the nucleic acids are present in a composition comprising pharmaceutically acceptable excipients for the parenteral, topical, cutaneous, vaginal, intranasal, subcutaneous or intra-ocular methods of administration.

46. A composition comprising a first nucleic acid comprising a sequence of a transgene of interest encoding a transcript of interest, and comprising a second nucleic acid comprising a sequence of an inhibitory transgene encoding an inhibitory transcript specific for said transcript of interest, wherein the sequences are each under the control of a transcriptional promoter, and wherein the activity of the inhibitory transcript is optionally regulated with at least one external agent and the activity of the transcript of interest is optionally regulated with at least one external agent.

47. The composition according to claim 46, wherein the inhibitory transcript is in the form of an antisense RNA which is complementary to at least one coding portion of the mRNA of the transgene of interest, and wherein the antisense RNA is capable of forming with the at least one coding portion of the mRNA of the transgene of interest a Watson and Crick-type linkage.

48. The composition according to claim 46, wherein the inhibitory transcript is in the form of an antisense RNA which is complementary to at least one noncoding portion of the mRNA of the transgene of interest, and wherein the antisense RNA is capable of forming with the at least one noncoding portion of the mRNA of the transgene of interest a Watson and Crick-type linkage.

49. The composition according to claim 48, wherein the inhibitory transcript is in the form of an antisense RNA which is complementary to at least one 5′ noncoding portion of the mRNA of the transgene of interest, and wherein the antisense RNA is capable of forming with the at least one 5′ noncoding portion of the mRNA of the transgene of interest a Watson and Crick-type linkage.

50. The composition according to claim 46, wherein the antisense RNA is at least 10 ribonucleotides long.

51. The composition according to claim 46, wherein the inhibitory transcript is in the form of an RNA capable of forming a triple helix with a portion of the nucleic acid comprising the sequence of the transgene of interest.

52. The composition according to claim 46, wherein the inhibitory transcript is in the form of a ribozyme.

53. The composition according to claim 46, wherein the sequence encoding the inhibitory transcript is under the control of a repressible promoter, and wherein at least one external agent causes the inhibition of said promoter.

54. The composition according to claim 53, wherein the promoter comprises at least one repeat of a tetracycline response operator, and at least one external agent is at least one external repressor agent chosen from tetracycline and tetracycline analogues.

55. The composition according to claim 46, wherein the nucleic acid encoding the inhibitory transcript also comprises an activatable autocatalytic aptamer sequence, and wherein the composition further comprises at least one external agent which is a specific ligand of the aptamer and is an activator of the aptamer.

56. The composition according to claim 46, wherein the nucleic acid encoding the inhibitory transcript also comprises a sequence which can be recognized by a ribozyme with ligand-dependent activity, and wherein the composition further comprises at least one external agent which is a ligand of the ribozyme and is an activator of the catalytic activity of the ribozyme.

57. The composition according to claim 46, wherein the sequence of the transgene of interest encoding the transcript of interest is placed under the control of an inducible promoter, and at least one external agent causes the activation of said promoter.

58. The composition according to claim 57, wherein the inducible promoter comprises a PPARα response element, and at least one external agent is a PPARα ligand.

59. The composition according to claim 58, wherein the inducible promoter comprises a PPARα response element, and at least one external agent is a PPARα ligand chosen from fibrates and fibrate analogues.

60. The composition according to claim 57, wherein the inducible promoter comprises a PPARγ response element, and at least one external agent is a PPARγ ligand.

61. The composition according to claim 60, wherein the inducible promoter comprises a PPARγ response element, and at least one external agent is a PPARγ ligand chosen from fatty acids and thiazolidinediones.

62. The composition according to claim 46, wherein the transgene of interest encodes a protein of therapeutic interest.

63. The composition according to claim 46, wherein the transgene of interest corrects a genetic abnormality or deficiency.

64 The composition according to claim 46, wherein the transgene of interest encodes an activator which is involved in the expression of another gene.

65. The composition according to claim 46, wherein the transgene of interest produces a transcript of interest having therapeutic activity.

66. The composition according to claim 46, wherein the nucleic acids are carried by a single vector or different vectors.

67. The composition according to claim 66, wherein the nucleic acids are carried on the same strand of the same vector.

68. The composition according to claim 66, wherein the vector is a plasmid, a cosmid, an artificial chromosome or a nonencapsidated DNA.

69. The composition according to claim 66, wherein the vector is a recombinant virus.

70. The composition according to claim 69, wherein the vector is a recombinant virus chosen from adenoviruses, retroviruses, herpesviruses, adeno-associated viruses, phages, and derivatives thereof.

71. The composition according to claim 66, wherein the vector is a bacterium or a parasite.

72. The composition according to claim 46, wherein the composition is in a form wherein the nucleic acids can be administered in vivo to the target tissue or cell using a physical or mechanical technique.

73. The composition according to claim 72, wherein the composition is in a form wherein the nucleic acids can be administered in vivo to the target tissue or cell using a physical or mechanical method chosen from injection, ballistic technique, electroporation, sonoporation, exposure to an electrical field, exposure to microwaves, exposure to heat or pressure, or by a combination of these techniques.

74. The composition according to claim 73, wherein the composition is in a form wherein the nucleic acids can be administered by injection, electrotransfer, or a combination thereof.

75. A method for regulating a transgene of interest in a target cell or tissue, comprising the composition as defined in claim 46.

77. The method according to claim 75, wherein the target cell or tissue is of nonhuman animal origin or human origin.

78. The method according to claim 75, wherein the target cell or tissue is a muscle cell or muscle tissue.

79. The method according to claim 75, wherein the target cell or tissue is of plant origin.

80. A pharmaceutical composition comprising the composition according to claim 46 and a suitable pharmaceutical excipient.

81. The pharmaceutical composition according to claim 80 in a form that can be injected into muscle, injected systemically, injected intra-arterially or injected intravenously.

82. The pharmaceutical composition according to claim 80 in a form that can be administered topically, cutaneously, orally, vaginally, intranasally, subcutaneously or parenterally.

83. The pharmaceutical composition according to claim 80, wherein the excipient is an excipient that can be injected into muscle, injected systemically, injected intra-arterially or injected intravenously, or the excipient can be administered topically, cutaneously, orally, vaginally, intranasally, subcutaneously, or parenterally.

84. A method for manufacturing a medicinal product intended for correcting a genetic abnormality or deficiency, comprising the composition according to claim 46.

85. A method of using the composition according to claim 46, for manufacturing a medicinal product intended for treating mitochondrial genetic diseases.

86. A method of using the composition according to claim 46, for manufacturing a medicinal product intended for treating myopathies.

87. A method of using the composition according to claim 46, for manufacturing a medicinal product intended for treating ischemias and stenosis.

88. A method of using the composition according to claim 46, for manufacturing a medicinal product intended for treating lysosomal diseases.

89. A method of using the composition according to claim 46, for manufacturing a medicinal product intended for treating hormonal disorders.

90. A method of using the composition according to claim 46, for manufacturing a medicinal product intended for treating hemophilia.

91. A method of using the composition according to claim 46, for manufacturing a medicinal product intended for treating inflammatory diseases or disorders.

92. The method according to claim 91, wherein the medicinal product is intended for treating the inflammatory disease chosen from rheumatoid arthritis.

93. A method of using the composition according to claim 46, for manufacturing a medicinal product intended for treating β-thalassemia.

94. A method of using the composition according to claim 46, for manufacturing a medicinal product intended to induce apoptosis.

95. A method of using the composition according to claim 46, for manufacturing a medicinal product intended for anticancer treatment.

96. A method of using the composition according to claim 46, for manufacturing a medicinal product intended for treating neurodegenerative diseases.

97. A method of using the composition according to claim 46, for manufacturing a medicinal product intended for treating cardiovascular diseases or disorders.

98. The method according to claim 97, wherein the medicinal product is intended for treating the cardiovascular disease or disorder chosen from hypertension.

99. A method of using the composition according to claim 46, for manufacturing a medicinal product intended for treating hyperlipidemias.

100. The method according to claim 99, wherein the medicinal product is intended for treating the hyperlipidemia chosen from obesity

101. A method of using the composition according to claim 46, for manufacturing vaccines.

102. A transgenic animal, wherein the animal carries a first nucleic acid comprising the sequence of a transgene of interest encoding a transcript of interest, and a second nucleic acid comprising the sequence of an inhibitory transgene encoding an inhibitory transcript specific for the transcript of interest, wherein each of the sequences are under the control of a transcriptional promoter, and the activity of the inhibitory transcript is optionally regulated with at least one external agent, and the activity of the transcript of interest is optionally regulated with at least one external agent.

103. The transgenic animal according to claim 102, wherein the inhibitory transcript is in the form of a genetic antisense RNA, of an RNA capable of forming a triple helix or of a ribozyme.

104. The transgenic animal according to claim 102, wherein the inhibitory transcript is regulated negatively via a repressible promoter, and at least one external agent causes the inhibition of said promoter.

105. The transgenic animal according to claim 102, wherein the inhibitory transcript also comprises an activatable autocatalytic aptamer sequence, and at least one external agent is a specific ligand of the aptamer sequence which is an activator of the aptamer sequence.

106. The transgenic animal according to claim 102, wherein the inhibitory transcript also comprises a sequence which can be recognized by a ribozyme with ligand-dependent activity, and at least one external agent is a ligand of the ribozyme which is an activator of the catalytic activity of the ribozyme.

107. The transgenic animal according to claim 102, wherein the transcript of interest is under the control of an inducible promoter, and at least one external agent causes the activation of said promoter.

108. A transgenic plant, wherein the plant carries a first nucleic acid comprising the sequence of a transgene of interest encoding a transcript of interest, and a second nucleic acid comprising the sequence of an inhibitory transgene encoding an inhibitory transcript specific for said transcript of interest, wherein each of the sequences are under the control of separate transcriptional promoters, and the activity of the inhibitory transcript is optionally regulated with at least one external agent, and the activity of the transcript of interest is optionally regulated with at least one external agent.

109. The transgenic plant according to claim 108, wherein the inhibitory transcript is in the form of a genetic antisense RNA, of an RNA capable of forming a triple helix or of a ribozyme.

110. The transgenic plant according to claim 108, wherein the inhibitory transcript is regulated negatively via a repressible promoter, and at least one external agent causes the inhibition of said promoter.

111. The transgenic plant according to claim 108, wherein the inhibitory transcript also comprises an activatable autocatalytic aptamer sequence, and at least one external agent is a specific ligand of this aptamer sequence.

112. The transgenic plant according to claim 108, wherein the inhibitory transcript also comprises a sequence which can be recognized by a ribozyme with ligand-dependent activity, and at least one external agent is a ligand of the ribozyme which is an activator of the catalytic activity of the ribozyme.

113. The transgenic plant according to claim 108, wherein the transcript of interest is under the control of an inducible promoter and at least one external agent causes the activation of said promoter.
